(19) United States
(12) Patent Application Publication
(10) Pub. No.: US 2024/0025946 A1
(43) Pub. Date: Jan. 25, 2024

(54) Cyclic Tetrapeptides and Metal Complexes Thereof

(71) Applicant: Universität Zürich, Zürich (CH)

(72) Inventors: Michal Shoshan, Zurich (CH); Mohammed Tagwa, Zurich (CH)

(21) Appl. No.: 18/039,530

(22) PCT Filed: Dec. 3, 2021
(86) PCT No.: PCT/EP2021/084278

§ 371 Date: May 31, 2023

(30) Foreign Application Priority Data
Dec. 3, 2020 (EP) 20211457.5

(51) Int. Cl. C07K 5/12; A61P 39/04

(52) U.S. Cl. CPC C07K 5/126; A61P 39/04; C07K 2319/00

(57) ABSTRACT

Provided herein is a cyclic tetrapeptides including alternating α- and 3-amino acids and metal complexes thereof. The cyclic tetrapeptides are useful for coordinating a metal selected from Pb, Cd, Hg and As. Also provided herein is the use of the cyclic tetrapeptides in treating a disease, particularly metal poisoning, and the use in remediation of contaminated water and soil. Also provided herein are methods for detecting said metals in various substrates are provided.

### CROSS-REFERENCE TO RELATED APPLICATIONS

This application is the United States national phase of International Application No. PCT/EP2021/084278 filed Dec. 3, 2021, and claims priority to European Patent Application No. 20211457.5 filed Dec. 3, 2020, the disclosures of each of which are hereby incorporated by reference in their entireties.

The Sequence Listing associated with this application is filed in electronic format via EFS-Web and is hereby incorporated by reference into the specification in its entirety. The name of the text file containing the Sequence Listing is 2304116_ST25.txt. The size of the text file is 5,842 bytes, and the file was created on Apr. 28, 2023.

### BACKGROUND OF THE INVENTION

### Field of the Invention

The present invention relates to cyclic tetrapeptides and metal complexes thereof. The cyclic tetrapeptides are suitable for coordinating a metal such as Pb, As, Cd and Hg. The invention further relates to the use of the cyclic tetrapeptides in the treatment of a disease, particularly metal poisoning, and in the diagnosis of said disease. Also, methods for removing or detecting said metal by applying the cyclic tetrapeptides to a substrate such as contaminated soil or water are provided.

### Description of Related Art

Toxic metals such as lead (Pb), arsenic (As), mercury (Hg) and cadmium (Cd) can be found in contaminated soil or water where they pose a risk to the ecosystem and health of living organisms. For example, toxic metals can enter the human body via contaminated drinking water. Furthermore, metals may accumulate in crops or animals in the food chain and may thus be ingested by humans.

Lead (Pb) is a non-essential, toxic metal considered the most harmful to human health. Pb poisoning is responsible for 1 million cases of death worldwide annually. Remarkably, every third child is poisoned by Pb, while even in the United States of America, above 3% of the children are found to have dangerous Pb concentrations in their blood.

The molecular mechanisms by which Pb is toxic are diverse and include interference with both cellular processes and organ functions. Under physiological conditions, Pb is predominantly found in its cationic state as Pb²⁺. that interacts with various proteins, primarily with the thiols of cysteine (Cys) and the carboxylates of aspartic (Asp) or glutamic (Glu) acids. This tight metal-binding alters the conformation of enzymes, resulting in diminished function. Pb²⁺. also substitutes several essential metal ions in metalloproteins, mainly calcium (Ca) and zinc (Zn) ions, causing protein dysfunction.

After uptake, Pb²⁺ is distributed to the soft tissues, with the liver and kidneys showing the highest accumulation levels. Due to its similar ionic radius as Ca²⁺, Pb²⁺ can also cross the blood-brain barrier, resulting in its accumulation in the brain. Finally, a significant fraction of Pb is stored in calcified tissues, and is released into the blood during pregnancy, becoming a source of exposure to the fetus while crossing the placenta.

Chelation therapy is the current treatment for Pb poisoning. It is based on administering a drug named a chelating agent (CA) that ideally should possess several essential characteristics: (a) low toxicity of the CA and the formed complex, (b) selectivity for the respective metal ion, (c) water solubility, (d) formation of an eliminable complex, and (e) ability to penetrate cells and tissues.

CAs used against Pb poisoning are predominantly ethylenediaminetetraacetic acid (EDTA) and dimercaptosuccinic acid (DMSA; FIG. 1).

These small-molecule drugs accomplish some of the requirements stated above. Yet, despite being the primary treatment for Pb poisoning, they suffer significant drawbacks, mainly low metal selectivity that results in essential metals being depleted from the body during treatment, which increases drug toxicity. Further, EDTA cannot cross cellular membranes, limiting its use to extracellular targets. They are also suspected of redistributing Pb²⁺ ions to the brain. As a result, these chelators are only approved for medicinal use in proven and extremely high toxic metals levels. However, critically, they are not approved for use in pregnant women, and only in rare pediatrics cases, even though these segments are among the most affected population.

Based on the above-mentioned state of the art, the objective of the present invention is to provide means and methods in the treatment of metal poisoning and its diagnosis as well as to detect and remove metals from substrates such as contaminated water or soil. This objective is attained by the subject-matter of the independent claims of the present specification, with further advantageous embodiments described in the dependent claims, examples, figures and general description of this specification.

### SUMMARY OF THE INVENTION

A first aspect of the invention relates to a compound of formula 1, particularly of formula 1a,

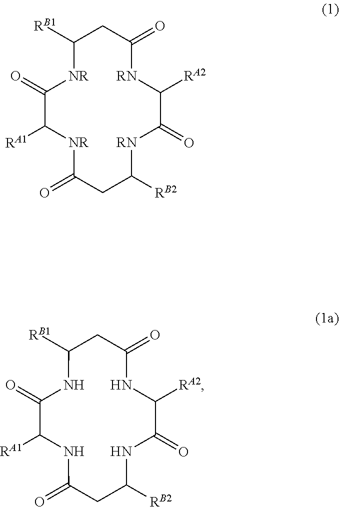

wherein
each R independently from any other R is independently selected from —CH₃ and —H, R^{A1} and R^{A2} are independently from each other a C₁₋₄-alkyl or phenyl, wherein the C₁₋₄-alkyl or phenyl is substituted by one or more substituents independently selected from —SH, —S—C₁₋₄-alkyl, —SeH, —Se—C₁₋₄-alkyl, —SO₃H, —COOH, —NH₂, —CONH₂, —NH—C(═NH)(NH₂) a 5- to 10-membered heterocycle, a cyclic hydrocarbon moiety comprising 3 to 10, particularly 3 to 6, carbon atoms, wherein
   the 5- to 10-membered heterocycle or the cyclic hydrocarbon moiety may optionally be substituted by one or more substituents selected from C₁₋₄-alkyl, —SH, (═S), —S—C₁₋₄-alkyl, —SeH, —Se—C₁₋₄-alkyl, —SO₃H, (═O), —COOH, —NH₂, —CONH₂, R^{B1} and R^{B2} are independently form each other
   —H, or
   a moiety selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a 5- to 10-membered heterocycle or a hydrocarbon moiety comprising 1 to 12 C atoms, wherein the 5- to 10-membered heterocycle or the hydrocarbon moiety is optionally substituted by one or more substituents independently selected from —OH, (═O), —SH, (═S), —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle, or
   a linker suitable for binding to a detectable marker or a solid support,
   a detectable marker, optionally linked by a linker, or
   a linker bound to a solid support.

The compound of formula 1 is a cyclic tetrapeptide consisting of two α-amino acids and two β-amino acids. The amino acids form a head-to-tail cyclization and may alternatively be represented by cyc-[Xaa-βXaa-Xaa-βXaa] (SEQ ID NO: 12), wherein Xaa depicts for an α-amino acid and βXaa depicts for a β-amino acid.

The cyclic tetrapeptide is suitable for binding a metal. Discrimination between toxic metals such as Pb²⁺ and other ions that are essential for human beings is achieved by a combination of the cavity size formed and the number and selection of the metal binding groups at R^{A} and R^{B}.

R^{A1} and R^{A2} of the α-amino acids contribute to metal binding. Particularly for the binding of Pb, R^{A1} and R^{A2} each comprise a soft or borderline binding moiety. Non-limiting examples for such moieties are thiol or carboxylic acid moieties, e.g. the thiol moiety of cysteine or the β-carboxylic acid moiety of aspartic acid.

R^{B1} and R^{B2} of the β-amino acids may fulfil various functions such as contributing to metal binding, mediating water solubility, facilitating cyclization during synthesis and stabilizing the ring structure and the metal complex.

If β-alanine is used for βXaa, i.e. R^{B} is H, intramolecular cyclization during synthesis is facilitated and the stability of the ring structure of the cyclic tetrapeptide is enhanced.

Water solubility of the cyclic tetrapeptide may be increased by using a moiety R^{B} that comprises a functional group such as an alcohol, an amide, carboxylic acid or a primary amine.

Enhancing the metal binding affinity may be achieved by additional coordination sites or by a second coordination sphere that is provided by suitable R^{B}. Also, the selectivity may be improved by steric control. For example, aliphatic or aromatic residues at R^{B1} and/or R^{B2} allow complex formation with smaller metal ions such as Hg.

Further functionalization of the cyclic tetrapeptide may be achieved by a linker, a linker bound to a solid support, or a detectable marker at R^{B1} and/or R^{B2}. Such cyclic tetrapeptides may be used in the diagnosis of metal poisoning, determining the degree of contamination of a substrate such as water or soil, or in the remediation of metal contaminated soil or water.

A second aspect of the invention relates to a metal complex consisting of a ligand and a metal, wherein the ligand is a compound according to the first aspect of the invention.

As described above, the compound according to the first aspect of the invention may bind to a metal via suitable moieties at R^{A} and R^{B}. For example, a thiol and/or carboxylic acid moiety in its deprotonated form may form a complex with Pb²*. The metal complex may comprise only one ligand (monomeric complex) or two ligands (dimeric complex).

A third aspect of the invention relates to the use of the compound according to the first aspect of the invention in the treatment of a disease.

A fourth aspect of the invention relates to the use of the compound according to the first aspect of the invention in the treatment of metal poisoning.

In another embodiment, the present invention relates to a pharmaceutical composition comprising at least one of the compounds of the present invention or a pharmaceutically acceptable salt thereof and at least one pharmaceutically acceptable carrier, diluent or excipient.

A fifth aspect of the invention relates to a method of determining whether a patient has, or is at risk of developing metal poisoning, comprising
a. determining the level of the metal using a compound according to the first aspect of the invention in an ex vivo blood, plasma or serum sample taken from the patient, and
b. establishing the statistical significance of the concentration of the metal.

Particularly compounds according to the first aspect of the invention that comprise a detectable marker, optionally linked by a linker, at R^{B} are suitable for the determination of the amount of metal in a sample.

A sixth aspect of the invention relates to a method of removing a metal from a substrate, wherein the method comprises using a compound according to the first aspect of the invention.

As described above, there is a constant need to remediate soil and water that are contaminated by metals such as Pb.

A seventh aspect of the invention relates to a method of detecting a metal in a substrate, wherein the method comprises using a compound according the first aspect of the invention. Particularly compounds according to the first aspect of the invention that comprise a detectable marker, optionally linked by a linker, at R^{B} are suitable for the detection of metals, e.g. Pb, in substrates such as contaminated water or soil.

### BRIEF DESCRIPTION OF THE DRAWINGS

FIG. 1 shows DMSA and EDTA as the benchmark drugs against Pb poisoning.

FIG. 2 shows detoxification ability of tested peptides compared to benchmark drugs and glutathione (GSH) at the highest administrated concentration in vivo on DH5a cells at 120 mM (10 equiv.; a) and in vitro on HT-29 cells at 10 mM (5 equiv.; b), concentration-dependent detoxification ability in HT-29 cells of 8 and the two drugs (c) and of EDTA and 8 as Ca vs. Na salts (d), toxicity in HT-29 cells of 8 and the two drugs (e). Values are mean±SD of >3 repeats each performed in triplicate.

FIGS. 3a and 3b show a metal complex consisting of Pb and cyc[Cys-βAla-Asp-βAla] as monomeric ligand (a) and cyc[Cys-βAla-Asp-βAla] as dimeric ligand (b).

FIG. 4 shows the dose-dependent recovery of HT-29 cells treated with Pb(NO₃)₂(2 mM) followed by the administration of Na₂8a, CaNa₂EDTA, and Na₂DMSA (1 h after the addition of Pb²⁺ ions; values are calculated relative to cells poisoned with Pb²⁺ ions as the negative control).

FIGS. 5a and 5b show (A) the average blood lead levels (BLL) and (B) urinary Pb of eight mice per group (in case of urine samples, only 34 out of 40 animals), collected at the experiment termination date (day 18) and analyzed by ICP-MS.

FIG. 6 shows peptides 1f (R═SH) and 8f (R═COOH) linked to a polystyrene tentagel resin.

FIG. 7 shows the Pb concentration as detected by ICP-MS and calculated compared to the original solution of two filtration rounds (dark gray) and one regeneration round (light gray) with EDTA in between of the negative control 0f and the two immobilized peptides 1f and 8f.

FIG. 8 shows the Pb concentration as detected by ICP-MS and calculated compared to the original solution of equimolar ZnCl₂+Pb(NO₃)₂ and CaCl₂+Pb(NO₃)₂ solutions and of human blood serum that was spiked with Pb(NO₃)₂(all salt solutions are at 25 mM) of 0f (black bars) 1f (light grey bars) and 8f (medium grey bars).

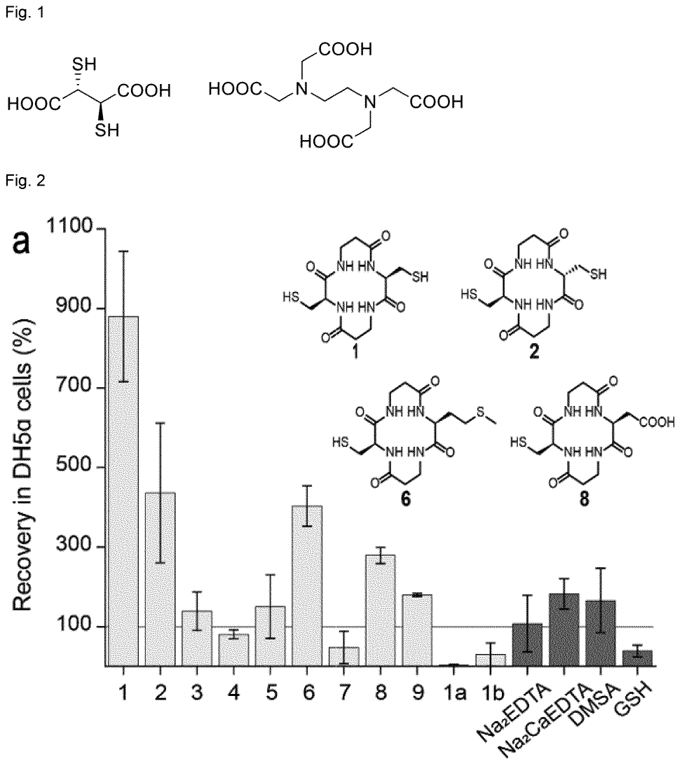

Terms and definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term “tetrapeptide” in the context of the present specification relates to a molecule consisting of 4 amino acids that form a linear chain wherein the amino acids are connected by peptide bonds. The tetrapeptide comprises two α-amino acids and two β-amino acids.

The term “cyclic tetrapeptide” relates to the tetrapeptide described above, wherein the amino acids form a head-to-tail cyclic as represented in formula 1.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions or “D” written before the amino acid name or amino acid code refer to the corresponding D- or (2R)-amino acids. Sequences are written left to right in the direction from the amino to the carboxy terminus. In accordance with standard nomenclature, α-amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, 1), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). The three letter or single letter code is also used after the Greek letter “s” for β-amino acids that comprise a residue at the β-carbon which is identical to the residue of the corresponding α-amino acid, e.g. “β-Ala” or “βAla” indicates the β-amino acid β-alanine. Homologues of α-amino acids or β-amino acids that differ by an additional methylene bridge (—CH₂—) in the side chain are referred to as “homo”-amino acid, e.g. homocysteine. “homo” is also abbreviated by “h”, e.g. hCys depicts for the α-amino acid homocysteine and “shGlu” depicts for β-homoglutamic acid.

In the context of the present invention, the term “5- to 10-membered heterocycle” relates to a compound that consists of 5 to 10 carbon atoms, wherein one or more carbon atoms are replaced by a heteroatom N, S or O, particularly N. Similarly, a “5- to 6-membered heterocycle” consists of 5 to 6 carbon atoms, wherein one or more carbon atoms are replaced by a heteroatom N, S or O, particularly N. The carbon atoms and one or more heteroatoms are connected by single and/or double bonds to form a ring structure. The ring structure may be monocyclic or bicyclic.

The term “hydrocarbon moiety comprising 3 to 10 carbon atoms” relates to a hydrocarbon moiety that comprises carbon-carbon single, double and/or triple bonds, particularly carbon-carbon single bonds and/or carbon-carbon double bonds. The carbon atoms may form a linear, branched or cyclic structure or combinations thereof.

The term alkyl refers to a linear or branched hydrocarbon moiety. A C₁₋₄-alkyl in the context of the present specification relates to a saturated linear or branched hydrocarbon having 1, 2, 3 or 4 carbon atoms. Similarly, a C₁₋₃-alkyl relates to a linear or branched hydrocarbon having up to 3 carbon atoms. Non-limiting examples for a C₁-C₄ alkyl include methyl, ethyl, propyl, n-butyl, 2-methylpropyl, tert-butyl. In certain embodiments, a C₁₋₄ alkyl refers to methyl (Me), ethyl (Et), propyl (Pr), isopropyl (iPr), n-butyl (Bu) and tertbutyl (tBu).

The term cyclic hydrocarbon moiety relates to a mono- or polycyclic hydrocarbon moiety that comprises carbon-carbon single, double and/or triple bonds, particularly carbon-carbon single bonds and/or carbon-carbon double bonds. The ring structures of a polycyclic hydrocarbon moiety may be bridged, fused or spirocyclic. Non-limiting examples for cyclic hydrocarbon moieties are aryls, e.g. phenyl, and cycloalkyls, e.g. cyclohexyl.

The term C₅₋₆-cycloalkyl in the context of the present specification relates to a saturated hydrocarbon ring having 5 or 6 carbon atoms.

The term fluorescent dye in the context of the present specification relates to a small molecule capable of fluorescence in the visible or near infrared spectrum.

### DESCRIPTION OF THE INVENTION

A first aspect of the invention relates to a compound of formula 1, particularly of formula 1a,

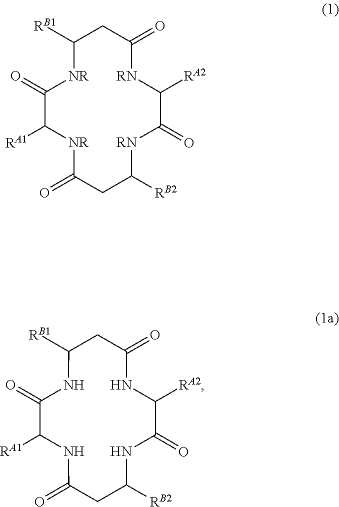

wherein
each R independently from any other R is independently selected from —CH₃ and —H, R^{A1} and R^{A2} are independently from each other a C₁₋₄-alkyl or phenyl, wherein the C₁₋₄-alkyl or phenyl is substituted by one or more substituents independently selected from —SH, —S—C₁₋₄-alkyl, —SeH, —Se—C₁₋₄-alkyl, —SO₃H, —COOH, —NH₂, —CONH₂, —NH—C(═NH)(NH₂) a five- to 10-membered heterocycle, a cyclic hydrocarbon moiety comprising 3 to 10, particularly 3 to 6, carbon atoms, wherein
   the 5- to 10-membered heterocycle or the cyclic hydrocarbon moiety may optionally be substituted by one or more substituents selected from C₁₋₄-alkyl, —SH, (═S), —S—C₁₋₄-alkyl, —SeH, —Se—C₁₋₄-alkyl, —SO₃H, (═O), —COOH, —NH₂, —CONH₂, particularly C₁₋₄-alkyl, —SH, —S—C₁₋₄-alkyl, —SeH, —Se—C₁₋₄-alkyl, —SO₃H, —COOH, —NH₂, —CONH₂,
R^{B1} and R^{B2} are independently form each other
   —H, or
   a moiety selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a five- to 10-membered heterocycle or a hydrocarbon moiety comprising 1 to 12 C atoms, wherein the 5- to 10-membered heterocycle or the hydrocarbon moiety is optionally substituted by one or more substituents independently selected from —OH, (═O), —SH, (═S), —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle, or
   a linker suitable for binding to a detectable marker or a solid support,
   a detectable marker, optionally linked by a linker, or
   a linker bound to a solid support.

In certain embodiments, at least one R is H and the other ones are —CH₃.

In certain embodiments, at least two R are H and the other ones are —CH₃.

In certain embodiments, at least three R are H and the other R is —CH₃.

In certain embodiments, at least one of the moieties R^{A1} and R^{A2} comprises a heteroatom S, N or O, particularly S. When the compound of formula 1 is used for binding a metal, the heteroatom at R^{A} forms a bond to the metal such as Pb, Hg, As and Cd, particularly Pb. Binding of Pb, Hg, As and Cd, particularly Pb, may not be achieved by a hydroxyl moiety such as in the side chain of serine. Thus, α-serine is not a suitable amino acid to provide suitable R^{A}. However, β-serine might still be used to provide a moiety R^{B} that enhances the water solubility of the cyclic tetrapeptide.

In certain embodiments, the compound is a compound of formula 2, 3, 4, 5, 6 or 7, particularly of formula 2a, 3a, 4a, 5a, 6a or 7a,

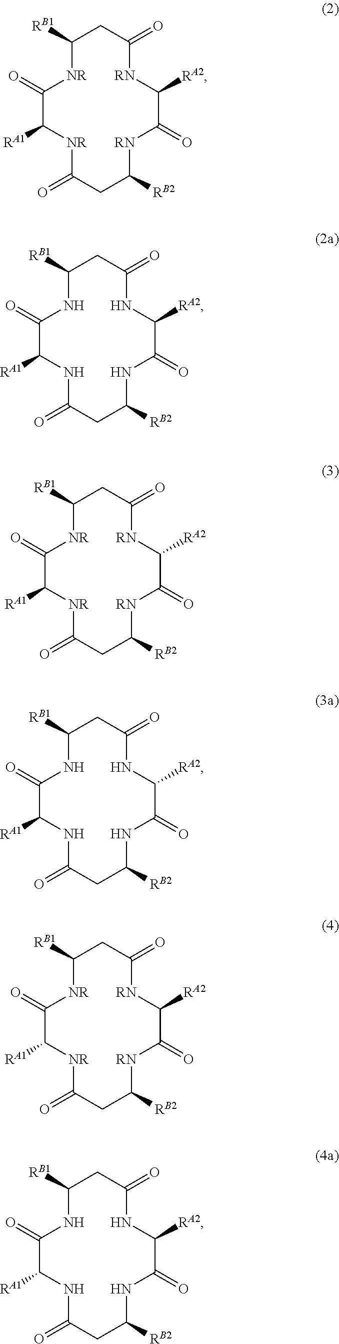

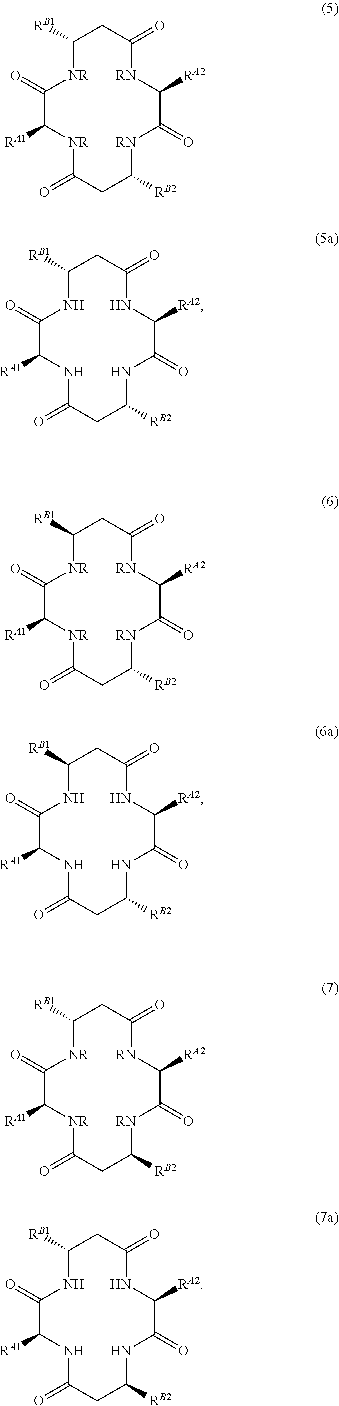

The cyclic tetrapeptides may be formed of L- or D-amino acids or a mix thereof. Due to economic reasons, particularly L-amino acids are used since they are usually cheaper than the corresponding D-amino acids.

For stable metal complex formation, the metal binding moieties R^{A1} and R^{A2} should orient the same direction, particularly for capturing Pb²⁺ in its favored unique hemidirected geometry.

In certain embodiments, the α-amino acids of the cyclic tetrapeptide are both L-amino acids or are both D-amino acids, particularly are both L-amino acids. This means, R^{A1} and R^{A2} are both bound to the α-carbon atom by an up-wedge bond or R^{A1} and R^{A2} are both bound to the α-carbon atom by a down-wedge bond.

In certain embodiments, the compound is a compound of formula 2, 5, 6 or 7, particularly of formula 2a, 5a, 6a or 7a.

In certain embodiments, the compound is a compound of formula 2 or 5, particularly of formula 2a or 5a.

In certain embodiments, the compound is a compound of formula 2, particularly of formula 2a.

In certain embodiments, R^{A1} and R^{A2} are independently from each other a C₁₋₄-alkyl, particularly a C₁₋₃-alkyl, more particularly a C₁₋₂-alkyl, substituted by one or more, particularly 1 or 2, substituents independently selected from —SH, —S—C₁₋₄-alkyl, —SeH, —Se—C₁₋₄-alkyl, —SO₃H, —COOH, —NH₂, —CONH₂, a five- to 10-membered heterocycle, a cyclic hydrocarbon moiety comprising 3 to 6 carbon atoms, wherein the 5- to 10-membered heterocycle or the cyclic hydrocarbon moiety may optionally be substituted by one or more, particularly 1, substituents selected from C₁₋₄-alkyl, —SH, (═S), —S—C₁₋₄-alkyl, —SeH, —Se—C₁₋₄-alkyl, —SO₃H, (═O), —COOH, —NH₂, —CONH₂, particularly C₁₋₄-alkyl, —SH, —S—C₁₋₄-alkyl, —SeH, —Se—C₁₋₄-alkyl, —SO₃H, —COOH, —NH₂, —CONH₂.

In certain embodiments, R^{A1} and R^{A2} are independently from each other a C₁₋₄-alkyl, particularly a C₁₋₃-alkyl, more particularly a C₁₋₂-alkyl, substituted by one or more, particularly 1 or 2, substituents independently selected from —SH, —S—C₁₋₄-alkyl, —SeH, —Se—C₁₋₄-alkyl, —SO₃H, —COOH, —NH₂, —CONH₂, a five- to 10-membered heterocycle, a cyclic hydrocarbon moiety comprising 3 to 6 carbon atoms, wherein the cyclic hydrocarbon moiety may optionally be substituted by one or more, particularly 1, substituents selected from C₁₋₄-alkyl, —SH, —S—C₁₋₄-alkyl, —SeH, —Se—C₁₋₄-alkyl, —SO₃H, —COOH, —NH₂, —CONH₂.

In certain embodiments, the heterocycle at R^{A1} and R^{A2} is selected from piperidinyl, piperazinyl, pyridinyl, pyrimidinyl, pyrazinyl, pyridazinyl, pyrrolyl, pyrazolyl, imidazolyl, mercaptoimidazolyl, thiofuranyl, oxazolonyl, indolyl, mercaptopurinyl, benzothiophenyl, particularly imidazolyl, mercaptoimidazolyl, thiofuranyl, indolyl, more particularly, mercaptoimidazolyl.

Thiofuran is also referred to as thiophene.

Benzothiophene is also referred to as benzothiofuran.

In certain embodiments, the heterocycle at R^{A1} and R^{A2} is selected from piperidinyl, piperazinyl, pyridinyl, pyrimidinyl, pyrazinyl, pyridazinyl, pyrrolyl, pyrazolyl, imidazolyl, mercaptoimidazolyl, thiofuranyl, oxazolonyl.

In certain embodiments, the heterocycle at R^{A1} and R^{A2} is selected from pyrrolyl, pyrazolyl, imidazolyl, mercaptoimidazolyl, thiofuranyl, oxazolonyl.

In certain embodiments, the heterocycle at R^{A1} and R^{A2} is selected from imidazolyl, mercaptoimidazolyl, thiofuranyl.

In certain embodiments, the heterocycle at R^{A1} and R^{A2} is selected from pyrrolyl, pyrazolyl, imidazolyl.

In certain embodiments, the heterocycle at R^{A1} and R^{A2} is selected from imidazolyl, indolyl.

In certain embodiments, the heterocycle at R^{A1} and R^{A2} is selected from imidazolyl.

In certain embodiments, the imidazolyl is 1H-imidazol-4-yl. For example, R^{A} is 1H-imidazol-4-yl when histidine is used as α-amino acid.

In certain embodiments, the indolyl is 1H-indol-3-yl. For example, R^{A} is 1H-indol-3-yl when tryptophan is used as α-amino acid.

In certain embodiments, the cyclic hydrocarbon moiety at R^{A1} and R^{A2} is selected from cyclopentyl, cyclohexyl and phenyl.

In certain embodiments, the cyclic hydrocarbon moiety at R^{A1} and R^{A2} is phenyl.

In certain embodiments, R^{A1} and R^{A2} are independently from each other a C₁₋₃-alkyl, particularly a C₁₋₂-alkyl, substituted by 1 or 2 substituents independently selected from —SH, —S—CH₃, —SeH, —Se—CH₃, —SO₃H, —COOH, —NH₂, —CONH₂, imidazolyl, indolyl and phenyl, wherein the phenyl may optionally be substituted by one or more, particularly 1, substituents selected from —SH, and —SeH, particularly —SH.

In certain embodiments, R^{A1} and R^{A2} are independently from each other a C₁₋₃-alkyl, particularly a C₁₋₂-alkyl, substituted by 1 or 2 substituents independently selected from —SH, —S—CH₃, —SeH, —Se—CH₃, —SO₃H, —COOH, —NH₂, —CONH₂.

In certain embodiments, R^{A1} and R^{A2} are independently from each other a C₁₋₃-alkyl, particularly a C₁₋₂-alkyl, substituted by 1 or 2 substituents independently selected from —SH, —S—CH₃, —SeH, —Se—CH₃, —SO₃H, —COOH, —NH₂, —CONH₂, imidazolyl, indolyl and phenyl, wherein the phenyl may optionally be substituted by one or more, particularly 1, substituents selected from —SH, and —SeH, particularly —SH.

In certain embodiments, R^{A1} and R^{A2} are independently selected from —CH₂—SH, —(CH₂)₂—SH, —CH₂—S—CH₃, —(CH₂)₂-S—CH₃, —CH(SH)(—CH₂—SH), —CH₂—CH(SH)(—CH₂—SH), —CH(SH)(—COOH), —CH(SH)—CH₂—COOH, —CH₂—CH(SH)(—COOH), -phenyl-SH, —CH₂—SO₃H, —(CH₂)₂-SO₃H —CH₂—COOH, —(CH₂)₂—COOH, —CH₂—NH₂, —(CH₂)₂-NH₂, —CH₂—CONH₂, —(CH₂)₂—CONH₂, —CH₂-imidazolyl, —CH₂-mercaptoimidazolyl and —CH₂-phenyl.

In certain embodiments, R^{A1} and R^{A2} are independently selected from —CH₂—SH, —(CH₂)₂—SH, —CH₂—S—CH₃, —(CH₂)₂—S—CH₃, —CH(SH)(—CH₂—SH), —CH₂—CH(SH)(—CH₂—SH), —CH(SH)(—COOH), —CH(SH)—CH₂—COOH, —CH₂—CH(SH)(—COOH), -phenyl-SH, —CH₂—SO₃H, —(CH₂)₂—SO₃H —CH₂—COOH, —(CH₂)₂—COOH, —CH₂—NH₂, —(CH₂)₂—NH₂, —CH₂—CONH₂, —(CH₂)₂—CONH₂.

In certain embodiments, R^{A1} and R^{A2} are independently selected from —CH₂—SH, —(CH₂)₂—SH, —(CH₂)₂—S—CH₃, —CH₂—CH(SH)(—CH₂—SH), —CH(SH)(—COOH), -phenyl-SH, —CH₂—SO₃H, —CH₂—COOH, —CH₂—NH₂, —CH₂—CONH₂, —CH₂-imidazolyl, and —CH₂-phenyl.

In certain embodiments, R^{A1} and R^{A2} are independently selected from —CH₂—SH, —(CH₂)₂—S—CH₃, —CH₂—COOH.

In certain embodiments,
R^{A1} and R^{A2} are identical and selected from a C₁₋₄-alkyl, particularly a C₁₋₃-alkyl, more particularly a C₁0.2-alkyl, substituted by one or more, particularly 1 or 2, substituents independently selected from —SH, —S—C₁₋₄-alkyl, —SeH, —Se—C₁₋₄-alkyl, —SO₃H, —COOH, —NH₂, —CONH₂, a 5- to 10-membered heterocycle, a cyclic hydrocarbon moiety comprising 3 to 6 carbon atoms, wherein
   the cyclic hydrocarbon moiety is substituted by one or more substituents, particularly 1 substituent, selected from —SH, —S—C₁₋₄-alkyl, —SeH, —Se—C₁₋₄-alkyl, —SO₃H, —COOH, —NH₂, —CONH₂, and wherein
   the 5- to 10-membered heterocycle is optionally substituted by one or more substituents, particularly 1 substituent, selected from C₁₋₄-alkyl, —SH, (═S), —S—C₁₋₄-alkyl, —SeH, —Se—C₁₋₄-alkyl, —SO₃H, (═O), —COOH, —NH₂, —CONH₂, particularly C₁₋₄-alkyl, —SH, —S—C₁₋₄-alkyl, —SeH, —Se—C₁₋₄-alkyl, —SO₃H, —COOH, —NH₂, —CONH₂, and/or
R^{A1} is selected from a C₁₋₄-alkyl, particularly a C₁₋₃-alkyl, more particularly a C₁₋₂-alkyl, substituted by 1 or 2 substituents selected from —SH, —S—C₁₋₄-alkyl, —SeH, —Se—C₁₋₄-alkyl and —COOH, particularly —SH, —S—C₁₋₄-alkyl and —COOH, and
R^{A2} is selected from a C₁₋₄-alkyl, particularly a C₁₋₃-alkyl, more particularly a C₁₋₂-alkyl, substituted by one or more, particularly 1 or 2, substituents independently selected from —SH, —S—C₁₋₄-alkyl, —SeH, —Se—C₁₋₄-alkyl, —SO₃H, —COOH, —NH₂, —CONH₂, a 5- to 10-membered heterocycle, a cyclic hydrocarbon moiety comprising 3 to 6 carbon atoms, wherein
   the 5- to 10-membered heterocycle or the cyclic hydrocarbon moiety may optionally be substituted by one or more substituents, particularly 1 substituent, selected from C₁₋₄-alkyl, —SH, (═S), —S—C₁₋₄-alkyl, —SeH, —Se—C₁₋₄-alkyl, —SO₃H, (═O), —COOH, —NH₂, —CONH₂, particularly C₁₋₄-alkyl, —SH, —S—C₁₋₄-alkyl, —SeH, —Se—C₁₋₄-alkyl, —SO₃H, —COOH, —NH₂, —CONH₂,
particularly R^{A2} is selected from a C₁₋₄-alkyl, particularly a C₁₋₃-alkyl, more particularly a C₁₋₂-alkyl, substituted by 1 or 2 substituents, particularly 1 substituent, independently selected from —SH, —S—C₁₋₄-alkyl, —SeH, —Se—C₁₋₄-alkyl, —COOH, —NH₂, —CONH₂, a five- to 6-membered heterocycle, particularly imidazolyl, mercaptoimidazolyl or thiofuranyl, a phenyl, particularly an unsubstituted phenyl,
   wherein the phenyl may optionally be substituted by one or more substituents, particularly 1 substituent, selected from —SH, —S—C₁₋₄-alkyl, —SeH, —Se—C₁₋₄-alkyl, wherein R^{A2} is selected in such a way that it differs from R^{A}.

In certain embodiments,
R^{A1} and R^{A2} are identical and selected from a C₁₋₃-alkyl, particularly a C₁₋₂-alkyl, substituted by 1 or 2 substituents independently selected from —SH, —S—CH₃, —SeH, —Se—CH₃, —SO₃H, —COOH, —NH₂, —CONH₂, imidazolyl, mercaptoimidazolyl, thiofuranyl, indolyl and phenyl, wherein the phenyl is substituted by one or more substituents, particularly 1 substituent, selected from —SH, and —SeH, particularly —SH, and/or
R^{A1} is selected from a C₁₋₃-alkyl, particularly a C₁₋₂-alkyl, substituted 1 or 2 substituents selected from —SH, —S—C₁₋₄-alkyl, —SeH, —Se—C₁₋₄-alkyl and —COOH, particularly —SH, —S—C₁₋₄-alkyl and —COOH, and
R^{A2} is selected from a C₁₋₃-alkyl, particularly a C₁₋₂-alkyl, substituted by 1 or 2 substituents independently selected from —SH, —S—CH₃, —SeH, —Se—CH₃, —SO₃H, —COOH, —NH₂, —CONH₂, imidazolyl, mercaptoimidazolyl, thiofuranyl, indolyl and phenyl,
   wherein the phenyl may optionally be substituted by one or more substituents, particularly 1 substituent, selected from —SH, and —SeH, particularly —SH,
particularly R^{A2} is selected from a C₁₋₃-alkyl, particularly a C₁₋₂-alkyl, substituted by 1 or 2 substituents, particularly 1 substituent, independently selected from —SH, —S—CH₃, —SeH, —Se—CH₃, —COOH, —NH₂, —CONH₂, imidazolyl and phenyl, particularly an unsubstituted phenyl or imidazoyl, wherein the phenyl may optionally be substituted by one or more substituents, particularly 1 substituent, selected from —SH, and —SeH, particularly —SH, wherein R^{A2} is selected in such a way that it differs from R^{A1}.

In certain embodiments,
R^{A1} and R^{A2} are identical and selected from —CH₂—SH, —(CH₂)₂—SH, —(CH₂)₂—S—CH₃, —CH₃—CH(SH)(—CH₂—SH), —CH(SH)(—COOH), -phenyl-SH, —CH₂—SO₃H, —CH₂—COOH and —CH₂-imidazolyl, and/or
R^{A1} is selected from —CH₂—SH and —CH(SH)(—COOH), and R^{A2} is selected from —CH₂—SH, —(CH₂)₂—SH, —(CH₂)₂—S—CH₃, —CH₂—COOH, —CH₂—NH₂, —CH₃—CONH₂, —CH₂-imidazolyl, and —CH₂-phenyl, wherein R^{A2} is selected in such a way that it differs from R^{A1}.

In certain embodiments, R^{A1} and R^{A2} are identical.

In certain embodiments of any aspect of the invention, the alkyl moiety of R^{A1} or of R^{A2} is not substituted by a 5- to 6-membered heterocycle or a cyclic hydrocarbon moiety.

In certain embodiments of any aspect of the invention, the alkyl moiety of R^{A1} or of R^{A2} is not substituted by a cyclic hydrocarbon moiety.

In certain embodiments, R^{B1} and R^{B2} are independently form each other
—H, or
a moiety selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a 5- to 10-membered heterocycle or a hydrocarbon moiety comprising 1 to 12 C atoms, wherein the 5- to 10-membered heterocycle or the hydrocarbon moiety is optionally substituted by one or more substituents independently selected from —OH, (═O), —SH, (═S), —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle.

In certain embodiments, R^{B1} and R^{B2} are independently selected from
—H, or
a moiety selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a five- to 10-membered heterocycle or a hydrocarbon moiety comprising 1 to 12 C atoms, wherein the hydrocarbon moiety is optionally substituted by one or more substituents independently selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle.

To enhance the water solubility of the cyclic tetrapeptide according to the invention, one and/or both moieties R^{B1} and R² may comprise a hydrophilic moiety. In certain embodiments, at least one of R^{B1} and R² is independently selected from a moiety selected from —OH, —COOH, —NH₂, —CONH₂, —SO₃H, a five- to 10-membered heterocycle or a hydrocarbon moiety comprising 1 to 12 C atoms, wherein the hydrocarbon moiety is optionally substituted by one or more substituents independently selected from —OH, —COOH, —NH₂, —CONH₂, —SO₃H, and a five- to 10-membered heterocycle.

To enhance the metal binding affinity, R^{B1} and R² may comprise a moiety that provides additional coordination sites and/or a second coordination sphere. In certain embodiments, R^{B1} and R² are independently form each other a moiety selected from —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a 5- to 10-membered heterocycle or a hydrocarbon moiety comprising 1 to 12 C atoms, wherein the 5- to 10-membered heterocycle or the hydrocarbon moiety is optionally substituted by one or more substituents independently selected from (═O), —SH, (═S), —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle. In certain embodiments, at least one of R^{B1} and R² is independently selected from —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a five- to 10-membered heterocycle or a hydrocarbon moiety comprising 1 to 12 C atoms, wherein the hydrocarbon moiety is optionally substituted by one or more substituents independently selected from —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle.

In certain embodiments, R^{B1} and R^{B2} are independently selected from
—H, or
a moiety selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a 5- to 10-membered heterocycle, a cyclopentyl, a cyclohexyl, phenyl or a C₁₋₄-alkyl, particularly C₁₋₄-alkyl, wherein the cyclopentyl, a cyclohexyl, phenyl or the C₁₋₄-alkyl, particularly C₁₋₄-alkyl, is optionally substituted by one or more substituents independently selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle, and wherein the 5- to 10-membered heterocycle is optionally substituted by one or more substituents independently selected from —OH, (═O), —SH, (═S), —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H.

In certain embodiments, the cyclopentyl, a cyclohexyl or phenyl at R^{B} is unsubstituted.

In certain embodiments, the heterocycle at R^{B1} and R^{B2} is selected from piperidinyl, piperazinyl, pyridinyl, pyrimidinyl, pyrazinyl, pyridazinyl, pyrrolyl, pyrazolyl, imidazolyl, particularly imidazolyl, mercaptoimidazolyl, thiofuranyl, oxazolonyl, indolyl, mercaptopurinyl, benzothiophenyl benzimidazolyl, quinolyl, isoquinolyl, diazanaphthalenyl.

In certain embodiments, the heterocycle at R^{B1} and R^{B2} is selected from piperidinyl, piperazinyl, pyridinyl, pyrimidinyl, pyrazinyl, pyridazinyl, pyrrolyl, pyrazolyl, imidazolyl, particularly imidazolyl, indolyl.

In certain embodiments, the heterocycle at R^{B1} and/or R^{B2} is defined as described for R^{A1} and R^{A2}. Reference is made to the respective embodiments relating to R^{A1} and R^{A2}.

In certain embodiments, R^{B1} and R^{B2} are independently selected from H, —C₃₋₆-alkyl, particularly —CH₂—CH(CH₃)₂, —CH₂-phenyl, —SH, —(CH₂)ₘ—SH, —(CH₂)ₘ—COOH and —(CH₂)ᵣ—CONH₂ with m and r being 0, 1, 2 or 3.

In certain embodiments, R^{B1} and R^{B2} are independently selected from H, —SH, —(CH₂)ₘ—SH, —(CH₂)ₘ—COOH and —(CH₂)ᵣ—CONH₂ with m and r being 0, 1, 2 or 3.

In certain embodiments, R^{B1} and R^{B2} are independently selected from H, —(CH₂)ₘ—COOH and —(CH₂)ᵣ—CONH₂ with m and r being 0, 1, 2 or 3.

In certain embodiments, R^{B1} and R^{B2} are independently selected from H, —(CH₂)ₘ—COOH and —CONH₂ with m being 1, 2 or 3.

In certain embodiments, m is 1, 2 or 3.

In certain embodiments, r is 0 or 1, particularly 1.

In certain embodiments, R^{B1} and R^{B2} are —H.

In certain embodiments, R^{B1} and R^{B2} are identical.

To facilitate detection by the cyclic tetrapeptide and/or a metal complex comprising the cyclic tetrapeptide according to the invention, the cyclic tetrapeptide may comprise a detectable marker.

In certain embodiments, the detectable marker is selected from a dye, an affinity tag, a magnetic bead, and a moiety comprising a radioisotope.

Suitable dyes are for example fluorescent dyes that are known to someone of skill in the art.

For detection of the cyclic tetrapeptide via an affinity tag, commonly known tags may be used.

Non-limiting examples for an affinity tag are strep-tag, glutathione-S-transferase (GST) tag, poly(His) tag.

In certain embodiments, the linker is a hydrocarbon moiety comprising up to 50 C atoms, particularly up to 20 C atoms, wherein one or more C atoms may optionally be replaced by 0, S or N.

In certain embodiments, the solid support is a resin, a bead, a surface of an electrode or the bottom/wall of a reaction vessel, particularly a surface of an electrode, a resin or a bead, more particularly a resin or a bead.

The compound according to the first aspect of the invention might be bound via a linker to a reaction vessel such as a 96-well plate or to a flow through device. This would facilitate the use of the compound in diagnostic/detection methods and the use of the compound in the remediation of contaminated water and soil, respectively.

In certain embodiments, the compound according to the first aspect of the invention is a compound of formula X1 to X22, particularly of formula X1-11 or X14-22,

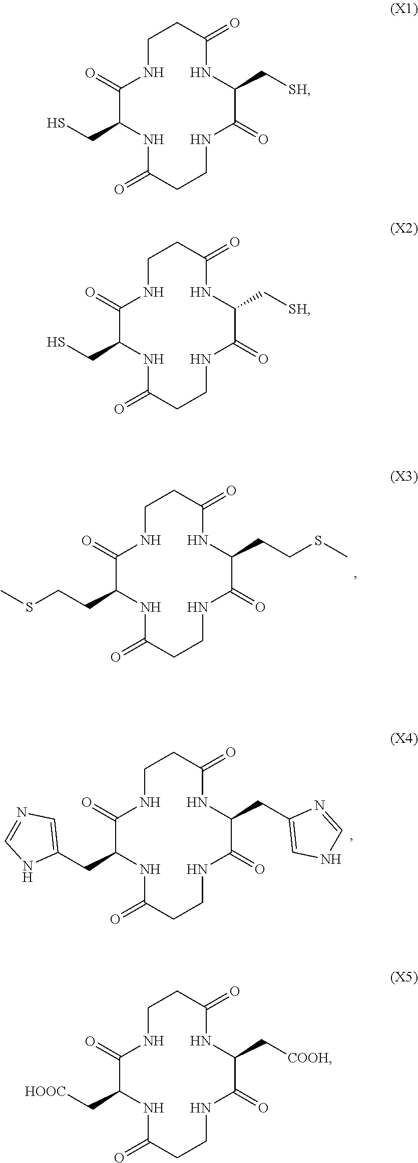

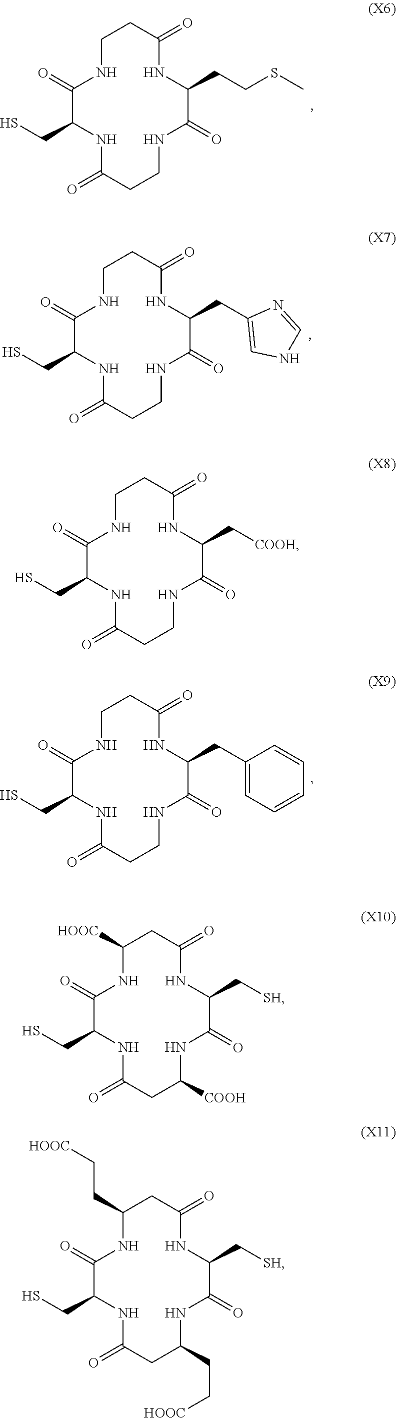 [SMILES: CSCC[C@@H]1NC(=O)CCNC(=O)[C@H](CS)NC(=O)CCNC1=O ; O=C1CCNC(=O)[C@H](Cc2c[nH]cn2)NC(=O)CCNC(=O)[C@H](CS)N1 ; O=C(O)C[C@@H]1NC(=O)CCNC(=O)[C@H](CS)NC(=O)CCNC1=O ; O=C1CCNC(=O)[C@H](Cc2ccccc2)NC(=O)CCNC(=O)[C@H](CS)N1 ; O=C1C[C@H](C(=O)O)NC(=O)[C@H](CS)NC(=O)C[C@H](C(=O)O)NC(=O)[C@H](CS)N1 ; O=C(O)CC[C@H]1CC(=O)N[C@@H](CS)C(=O)N[C@@H](CCC(=O)O)CC(=O)N[C@@H](CS)C(=O)N1]

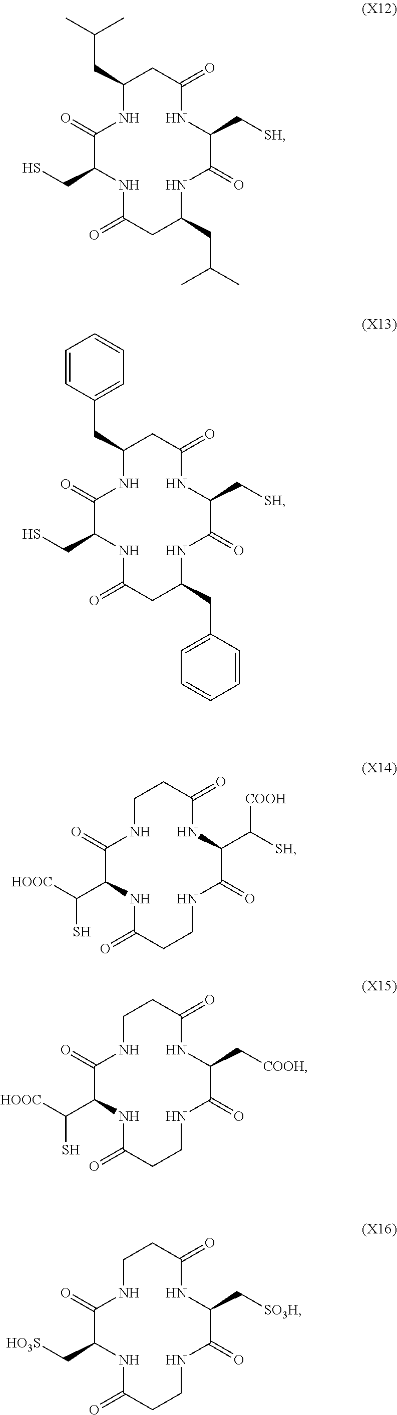 [SMILES: CC(C)C[C@H]1CC(=O)N[C@@H](CS)C(=O)N[C@@H](CC(C)C)CC(=O)N[C@@H](CS)C(=O)N1 ; O=C1C[C@H](Cc2ccccc2)NC(=O)[C@H](CS)NC(=O)C[C@H](Cc2ccccc2)NC(=O)[C@H](CS)N1 ; O=C1CCNC(=O)[C@H](C(S)C(=O)O)NC(=O)CCNC(=O)[C@H](C(S)C(=O)O)N1 ; O=C(O)C[C@@H]1NC(=O)CCNC(=O)[C@H](C(S)C(=O)O)NC(=O)CCNC1=O ; O=C1CCNC(=O)[C@H](CS(=O)(=O)O)NC(=O)CCNC(=O)[C@H](CS(=O)(=O)O)N1]

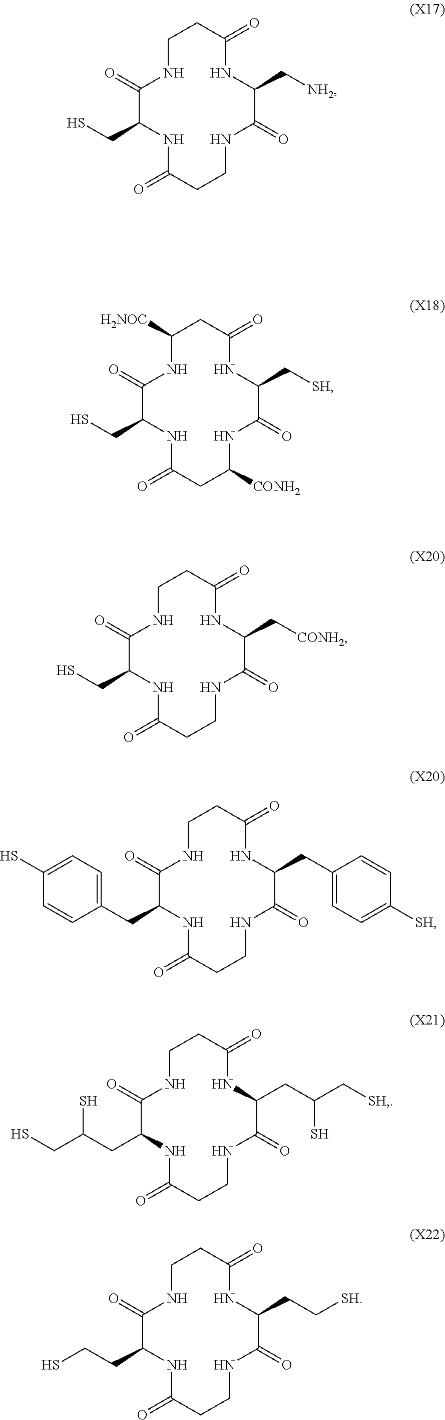 [SMILES: NC[C@@H]1NC(=O)CCNC(=O)[C@H](CS)NC(=O)CCNC1=O ; NC(=O)[C@H]1CC(=O)N[C@@H](CS)C(=O)N[C@@H](C(N)=O)CC(=O)N[C@@H](CS)C(=O)N1 ; NC(=O)C[C@@H]1NC(=O)CCNC(=O)[C@H](CS)NC(=O)CCNC1=O ; O=C1CCNC(=O)[C@H](Cc2ccc(S)cc2)NC(=O)CCNC(=O)[C@H](Cc2ccc(S)cc2)N1 ; O=C1CCNC(=O)[C@H](CC(S)CS)NC(=O)CCNC(=O)[C@H](CC(S)CS)N1 ; O=C1CCNC(=O)[C@H](CCS)NC(=O)CCNC(=O)[C@H](CCS)N1]

In certain embodiments of any aspect of the invention described herein, R^{A1} and R^{A}2 are not —CH₂-imidazolyl, and R^{A1} and R^{A}2 are not —CH₂-phenyl.

In certain embodiments of any aspect of the invention described herein, R^{A1} and R^{A}2 are not —CH₂-imidazoly.

In certain embodiments of any aspect of the invention described herein, R^{A1} and R^{A}2 are not —CH₂-phenyl.

In certain embodiments of any aspect of the invention described herein, the compound of formula 1 is not a compound of formula D1 or D2,

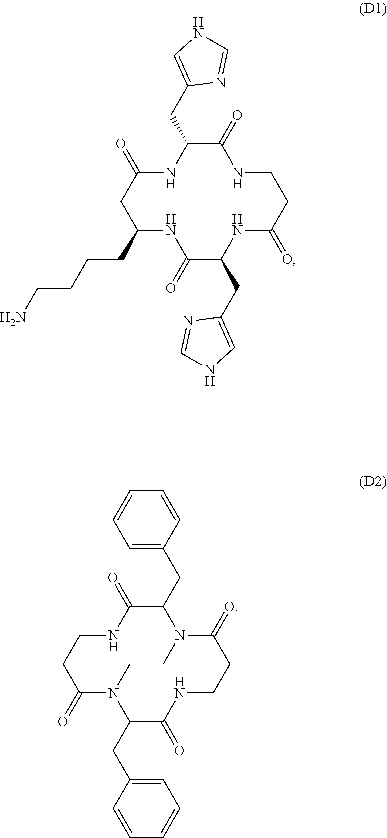 [SMILES: NCCCC[C@H]1CC(=O)N[C@H](Cc2c[nH]cn2)C(=O)NCCC(=O)N[C@@H](Cc2c[nH]cn2)C(=O)N1 ; CN1C(=O)CCNC(=O)C(Cc2ccccc2)N(C)C(=O)CCNC(=O)C1Cc1ccccc1]

A second aspect of the invention relates to a metal complex consisting of a ligand and a metal, wherein the ligand is a compound according to the first aspect of the invention.

As described above, the compound according to the first aspect of the invention may bind to a metal via suitable moieties at R^{A} and R^{B}.

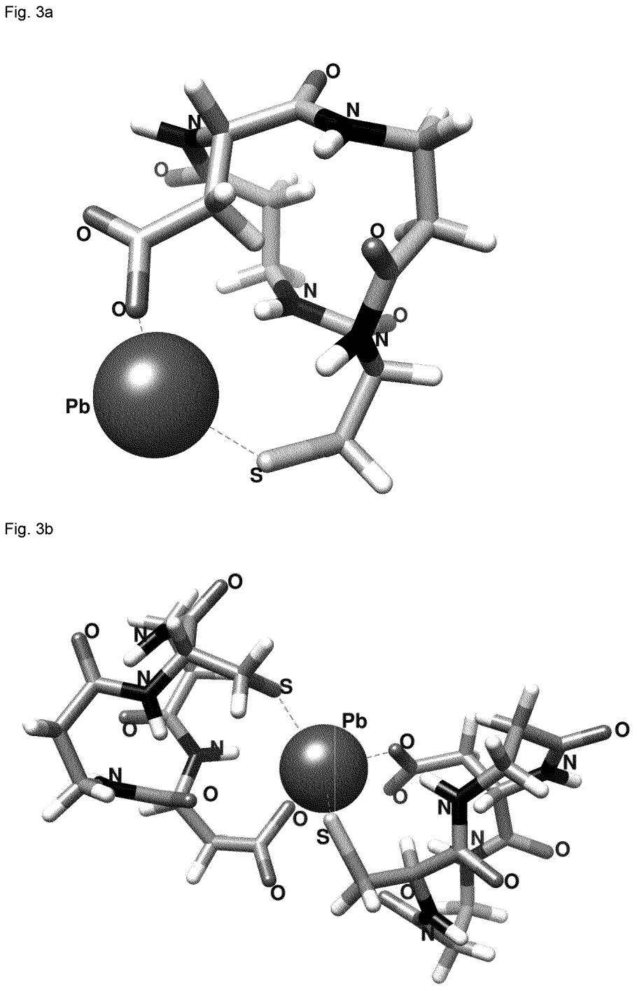

In certain embodiments, the binding moiety of the compound according to the first aspect of the invention binds to the metal in its deprotonated from. For example, a thiol and/or carboxylic acid moiety in its deprotonated form may form a complex with Pb²⁺ as shown below (see also FIG. 3):

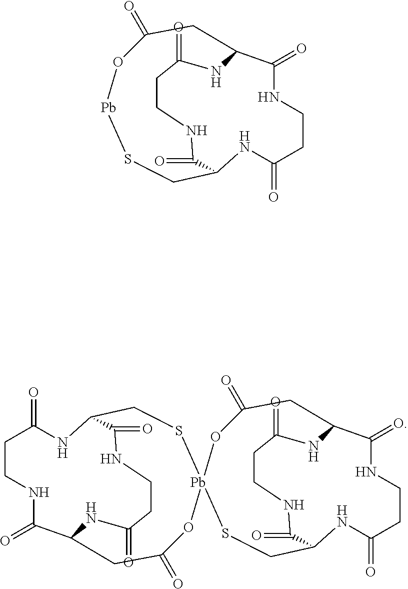

In certain embodiments, the ligand is an anion.

Usually the ratio of metal to peptide is 1:1 or 1:2, i.e. the complex is monomeric or dimeric.

In certain embodiments, the complex is dimeric, particularly homodimeric.

In certain embodiments, the metal is selected from Pb, As, Cd and Hg, in particular the metal is Pb.

With regard to the ligand, reference is made to the embodiments of the first aspect of the invention.

A third aspect of the invention relates to the use of the compound according to the first aspect of the invention in the treatment of a disease.

In certain embodiments, the compound according to the first aspect of the invention is for use in the treatment of a disease.

With regard to the compound, reference is made to the embodiments of the first aspect of the invention.

A fourth aspect of the invention relates to the use of the compound according to the first aspect of the invention in the treatment of metal poisoning.

In certain embodiments, the compound according to the first aspect of the invention is for use in the treatment of metal poisoning.

In certain embodiments, the metal poisoning is selected from Pb poisoning, As poisoning, Cd poisoning and Hg poisoning.

In certain embodiments, the metal poisoning is Pb poisoning.

In a medical context, the compound according to the first aspect of the invention may be applied by standard methods as described in Sears, M.E. (2013).

With regard to the compound, reference is made to the embodiments of the first aspect of the invention.

A fifth aspect of the invention relates to a method of determining whether a patient has, or is at risk of developing metal poisoning, particularly Pb poisoning, As poisoning, Cd poisoning and Hg poisoning, more particularly Pb poisoning comprising
a. determining the level of the metal, particularly of Pb, As, Cd and/or Hg using a compound according to the first aspect of the invention in an ex vivo blood, plasma or serum sample taken from the patient, and
b. establishing the statistical significance of the concentration of the metal.

Particularly compounds according to the first aspect of the invention that comprise a detectable marker, optionally linked by a linker, at R^{B} are suitable for the determination of the amount of metal in a sample.

Statistical significance might be established by determining the ratio of free ligand, i.e. the compound according to the first aspect of the invention, to the metal complex. The signal obtained when detecting the marker, may be compared to a standard.

With regard to the compound, reference is made to the embodiments of the first aspect of the invention.

The invention further encompasses the use of a compound according to the first aspect of the invention for use in the manufacture of a kit for the detection of developing metal poisoning, particularly Pb poisoning, As poisoning, Cd poisoning and Hg poisoning, more particularly Pb poisoning.

Wherever alternatives for single separable features are laid out herein as “embodiments”, it is to be understood that such alternatives may be combined freely to form discrete embodiments of the invention disclosed herein. Thus, any of the alternative embodiments for a detectable label may be combined with any of the alternative embodiments of ligand/compound according to the first aspect of the invention and these combinations may be combined with any medical indication or diagnostic method mentioned herein.

A sixth aspect of the invention relates to a method of removing a metal, particularly a metal selected from Pb, As, Cd and Hg, more particularly Pb, from a substrate, particularly soil or an aqueous solution or aqueous suspension, wherein the method comprises using a compound according to the first aspect of the invention.

Particularly compounds according to the first aspect of the invention that comprise a detectable marker, e.g. an affinity tag, or that are bound via a linker to a solid support are suitable for this method.

With regard to the compound, reference is made to the embodiments of the first aspect of the invention.

A seventh aspect of the invention relates to a method of detecting a metal, particularly a metal selected from Pb, As, Cd and Hg, more particularly Pb, in a substrate, particularly soil or an aqueous solution or aqueous suspension, wherein the method comprises using a compound according the first aspect of the invention.

Particularly compounds according to the first aspect of the invention that comprise a detectable marker, optionally linked by a linker, at R^{B} are suitable for the determination of the amount of metal in a sample.

With regard to the compound, reference is made to the embodiments of the first aspect of the invention.

Another aspect of the invention relates to the preparation of the compound according to the first aspect of the invention. The preparation comprises the following steps:
Providing a tetrapeptide consisting of two α-amino acids Xaa and two β-amino acids βXaa, wherein the tetrapeptide is characterized by the sequence βXaa-Xaa-βXaa-Xaa (SEQ ID NO: 13) or Xaa-βXaa-Xaa-βXaa (SEQ ID NO: 1414) from N- to C-terminus, particularly βXaa-Xaa-βXaa-Xaa (SEQ ID NO: 13),
adding a coupling reagent and a base yielding a reaction mix,
in a diluting step, diluting the reaction mix in an organic solvent, particularly CH₂Cl₂ or DMF, more particularly CH₂Cl₂.

In certain embodiments, the coupling reagent is selected from PyBOP, HATU (1-[bis(dimethylamino)methylene]-1H-1,2,3-triazolo[4,5-b]pyridinium 3-oxide hexafluorophosphate, CAS No. 148893-10-1), HCTU (O-(1H-6-chlorobenzotriazole-1-yl)-1,1,3,3-tetramethyluronium hexafluorophosphate, CAS No. 330645-87-9), HOBt/DIC (benzotriazol-1-ol , CAS No. 2592-95-2) and N,N′-di(propan-2-yl)methanediimine, CAS No. 693-13-0), DCC (N,N′-dicyclohexylmethanediimine, CAS No. 538-75-0), DPPA (diphenyl phosphorazidate, CAS No. 26386-88-9).

In certain embodiments, the coupling reagent is PyBOP. The term “PyBOP” relates to benzotriazol-1-yl-oxytripyrrolidinophosphonium hexafluorophosphate (CAS No. 128625-52-5).

In certain embodiments, 1 to 2 mole equivalents of the coupling reagent in relation to the mole amount of tetrapeptide are used.

In certain embodiments, 1.5 mole equivalents in relation to the mole amount of tetrapeptide are used.

In certain embodiments, the base is Hunig's base. The term “Hunig's base” relates to N-Ethyl-N-(propan-2-yl)propan-2-amine (CAS No. 7087-68-5).

In certain embodiments, 2 to 6 mole equivalents of base in relation to the mole amount of tetrapeptide are used.

In certain embodiments, 3 mole equivalents of base in relation to the mole amount of tetrapeptide are used.

In certain embodiments, the concentration of the tetrapeptide in the diluting step is 0.01 mM to 10 mM, particularly 0.05 mM to 2 mM.

In certain embodiments, the concentration of the tetrapeptide in the diluting step is 0.1 mM.

In certain embodiments, the dilution step is performed for 12 h to 72 h, particularly for 16 h to 48 h.

In certain embodiments, the diluting step is followed by an evaporation step. To allow fast evaporation, a solvent with a low boiling point such as CH₂Cl₂ may be used. When the boiling point of the solvent, e.g. DMF, is higher, evaporation may become tedious.

In certain embodiments, the method is performed at a temperature ranging from 15° C. to 40° C., particularly ranging from 20° C. to 25° C. The method may be performed at ambient temperature. There is no need to heat or cool down the reaction mixture.

The tetrapeptide may comprise protecting groups. Suitable protecting groups as well as methods for deprotection are known to one of skill in the art.

Medical treatment, Dosage Forms and Salts

Similarly, within the scope of the present invention is a method for treating metal poisoning, particularly Pb poisoning, As poisoning, Cd poisoning and Hg poisoning, more particularly Pb poisoning, in a patient in need thereof, comprising administering to the patient a compound according to the first aspect of the invention.

Similarly, a dosage form for the prevention or treatment of metal poisoning, particularly Pb poisoning, As poisoning, Cd poisoning and Hg poisoning, more particularly Pb poisoning is provided, comprising a compound according to any of the above aspects or embodiments of the invention.

The skilled person is aware that any specifically mentioned drug compound mentioned herein may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms.

Optionally, a pharmaceutically acceptable carrier and/or excipient may be present.

Topical administration is also within the scope of the advantageous uses of the invention. The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transdermal Drug Delivery Systems: Revised and Expanded (2^{nd} Ed., CRC Press 2002, ISBN-13: 978-0824708610); Osborne and Amann (Eds.): Topical Drug Delivery Formulations (1^{st} Ed. CRC Press 1989; ISBN-13: 978-0824781835).

Pharmaceutical Compositions and Administration

Another aspect of the invention relates to a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant and easily handleable product.

In embodiments of the invention relating to topical uses of the compounds of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The pharmaceutical composition can be formulated for enteral administration, particularly oral administration or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The pharmaceutical composition can be formulated for parenteral administration, for example by i.v. infusion, intradermal, subcutaneous or intramuscular administration.

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

Method of Manufacture and Method of Treatment according to the invention

The invention further encompasses, as an additional aspect, the use of a compound according to the first aspect of the invention, or its pharmaceutically acceptable salt, as specified in detail above, for use in a method of manufacture of a medicament for the treatment or prevention of metal poisoning, particularly Pb poisoning, As poisoning, Cd poisoning and Hg poisoning, more particularly Pb poisoning.

Similarly, the invention encompasses methods of treatment of a patient having been diagnosed with a disease associated with metal poisoning, particularly Pb poisoning, As poisoning, Cd poisoning and Hg poisoning, more particularly Pb poisoning. This method entails administering to the patient an effective amount of compound according to the first aspect of the invention, or its pharmaceutically acceptable salt, as specified in detail herein.

The invention is further illustrated by the following examples and figures, from which further embodiments and advantages can be drawn. These examples are meant to illustrate the invention but not to limit its scope.

### EXAMPLES

### Example 1: Synthesis of Cyclic Tetrapeptides

For compounds described in this example, a scaffold composed of the sequence cyc-[Xaa-βAla-Xaa-βAla] (SEQ ID NO: 15) (Xaa depicts for any α-AA; Scheme 1) was chosen, where in addition to enhancing stability, βAla was expected to facilitate the challenging intramolecular cyclization of the tetrapeptide.

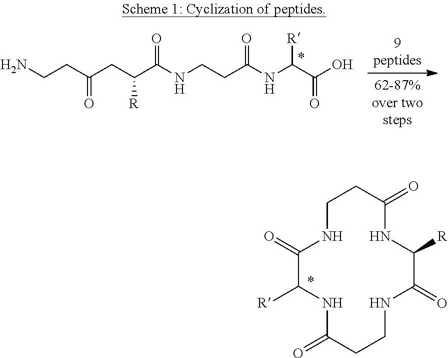

Herein the inventors present a family of cyclic tetrapeptides that were designed to detoxify Pb²⁺ ions. The peptides were examined for their ability to recover Pb-exposed bacteria and human cells, where one particular peptide (8) excelled to a greater extent than the benchmark chelating agents (CA). Mechanistic studies of the successful peptide shed light on its biological outcome and medicinal potential.

The inventors started their studies by synthesizing nine sidechain-protected linear peptides (Table 1, 1-9). Typically, head-to-tail cyclization occurs in dimethylformamide (DMF) as a solvent and only rarely of peptides shorter than pentamers (White et al, 2011). The inventors aimed at cyclizing tetrapeptides in the absence of a high boiling-point solvent as DMF. Upon condition screening, the inventors found the following ideal conditions: PyBOP and Hunig's base (1.5 and 3.0 equivalents, respectively) as the coupling reagent and the base, respectively, and ultrahigh dilution of the peptide (0.1 mM) in CH₂Cl₂ for 16-48 h until full conversion was obtained. The cyclic peptides were then side-chain deprotected and purified without the need for HPLC, reaching above 95% purity and with a yield of 62-87% over two steps (cyclization and deprotection). HR-ESI-MS and ¹H and ¹³C NMR indicated exclusively intramolecular cyclization to form the desired tetramers.

TABLE 1
Peptides and benchmark compounds studied

| | | | | | | | Toxicity^{c,e} | | |
|---|---|---|---|---|---|---|---|---|---|
| | | Recovery^{c,d} | | | | | | Maximal | |
| Name | | Aqueous | | | | | IC₅₀^{e} | inhibition^{f} | |
| (SEQ ID NO) | Sequenceª | solubility | DH5α (%) | | HT-29 (%) | | (mM) | (%) | |
| 1 | [Cys-βAla]₂ | insoluble | 879 | (164) | — | | — | — | |
| (SEQ ID NO: 1) | | | | | | | | | |
| 2 | Cys-βAla- | insoluble | 436 | (176) | — | | — | — | |
| (SEQ ID NO: 2) | DCys-βAla | | | | | | | | |
| 3 | [Met-βAla]₂ | insoluble | 139 | (48) | — | | — | — | |
| (SEQ ID NO: 3) | | | | | | | | | |
| 4 | [His-βAla]₂ | Soluble | 80 | (11) | 106 | (3) | 5.8 (0.7) | 62 | (3) |
| (SEQ ID NO: 4) | | | | | | | | | |
| 5 | [Asp-βAla]₂ | soluble^{b} | 150 | (80) | 121 | (16) | n.d.^{g} | 44 | (8) |
| (SEQ ID NO: 5) | | | | | | | | | |
| 6 | Cys-βAla- | insoluble | 403 | (51) | — | | — | — | |
| (SEQ ID NO: 6) | Met-βAla | | | | | | | | |
| 7 | Cys-βAla- | insoluble | 47 | (41) | — | | — | — | |
| (SEQ ID NO: 7) | His-βAla | | | | | | | | |
| 8 | Cys-βAla- | soluble^{b} | 279 | (21) | 334 | (42) | n.d.^{g} | 15 | (5) |
| (SEQ ID NO: 8) | Asp-βAla | | | | | | | | |
| 9 | Cys-βAla- | insoluble | 180 | (4) | — | | — | — | |
| (SEQ ID NO: 9) | Phe-βAla | | | | | | | | |
| 1a | [Cys-βAsp]₂ | soluble^{b} | 2 | (2) | 106 | (10) | n.d.^{g} | 0 | (1) |
| (SEQ ID NO: 10) | | | | | | | | | |
| 1b | [Cys-βhGlu]₂ | soluble^{b} | 30 | (29) | — | | — | — | |
| (SEQ ID NO: 11) | | | | | | | | | |
| Na₂EDTA | | — | 107 | (72) | 41 | (6) | 0.6 (0.2) | 72 | (4) |
| Na₂CaEDTA | | — | 182 | (39) | 95 | (16) | 3.9 (0.4) | 63 | (11) |
| DMSA | | — | 165 | (81) | 110 | (10) | 3.7 (0.1) | 77 | (3) |
| GSH^{h} | | — | 39 | (15) | 126 | (9) | 9.6 (1.1) | 57 | (8) |

^{a}three-letter code of AAs, h depicts for homo;
^{b}with the addition of 2 equiv. NaOH or 1 equiv. of Ca(OH)₂;
^{c}values are mean ± SD of >3 repeats each performed in triplicate;
^{d}presented values are at the highest concentrations;
^{e}the concentration at 50% viability;
^{f}the inhibition at the highest concentration;
^{g}not detected as is it too high;
^{h}glutathione

### Example 2: In Vivo and In Vitro Detoxification

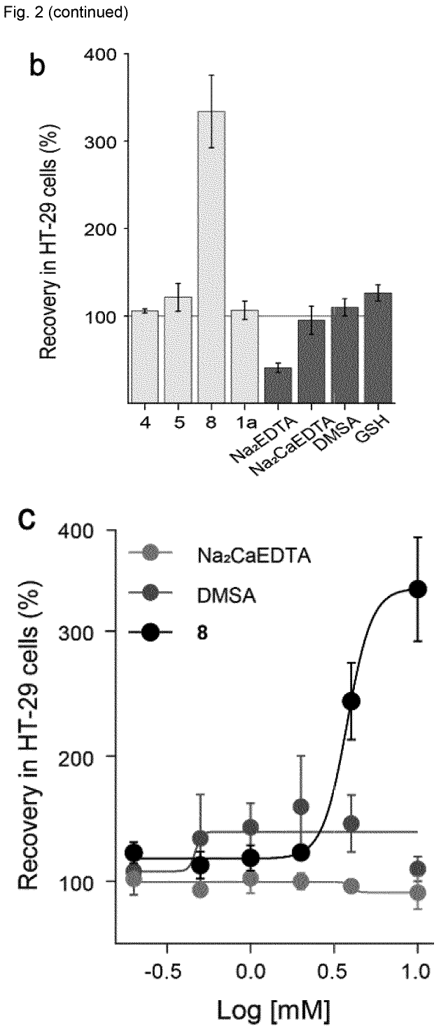

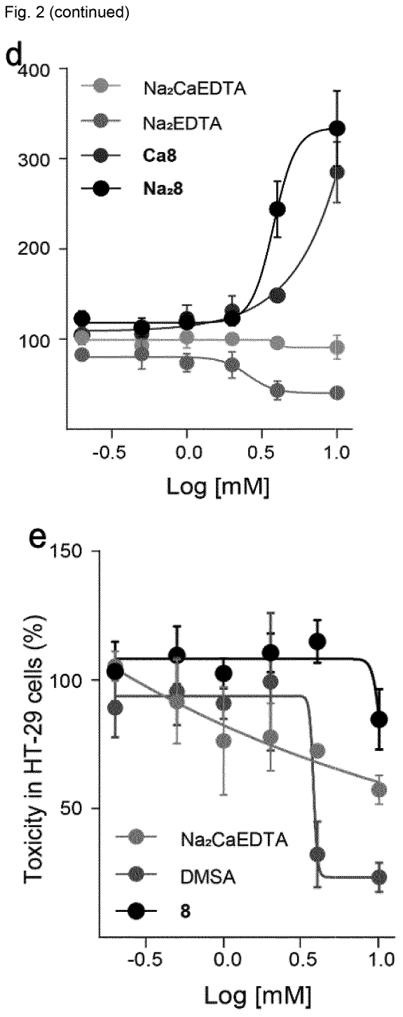

Desalted peptides were then assessed for their ability to detoxify Pb (FIGS. 2a-d). The inventors designed two assays for rapid and reliable screening of potential CAs both in vivo on bacteria and then in vitro on human cell culture. Briefly, DH5a or HT-29 cells were first exposed to Pb(NO₃)₂ slightly below the minimal inhibitory concentration and then treated with various concentrations of the investigated CA, ranging from 0.1 to 10 equivalents. The viability of the cells was determined by colony counting or by crystal violet (Feoktistova et al, 2016), for the bacteria and the human cells, respectively, and was compared to poisoned cells that were not treated with any CA as the negative control. Performing both assays proved highly valuable, mainly since the in vivo assay examines CAs on a solidified medium, eliminating any limitations derived from low solubility of the compounds. On the other hand, testing the compounds on human cells cannot be performed with insoluble compounds but is more relevant for medicinal purposes.

Within the nine peptides, four exhibited outstanding results in detoxifying poisoned E. coli compared to the benchmark compounds (FIG. 2a), all of which contain at least one Cys and an additional residue that can bind Pb²⁺; Cys, DCys, Met, and Asp for peptides 1, 2, 6, and 8, respectively. Treatment with 1 increased the recovery more than 8-fold while substituting one of the LCys with DCys reduced the detoxification ability of peptide 2. This points to the requirement that the binding moieties should orient the same direction, capturing Pb²⁺ in its favored unique hemidirected geometry. Surprisingly, the homo-functionalized peptides 3-5 showed insufficient activity, indicating a poor metal selectivity or low Pb affinity. Noteworthy, the linear analogs of peptides 1-8 were also tested in vivo, showing in all cases almost no detoxification ability. The inventors concluded that in addition to an expected enhanced proteolytic stability, the cyclization also fosters a preorganization of the ligand that endows its metal affinity by improving the coordination properties.

Despite being highly active in vivo, peptides 1, 2, and 6 showed low aqueous solubility, reducing their effectiveness as potential CAs. Attempts to solubilize them, including in different pH conditions, formulation with PEG or co-solvent systems with DMSO failed. Therefore, two analogs of 1 where βAla is substituted by PAsp or PhGlu, to form peptides 1a and 1b, respectively, were synthesized. These peptides proved high solubility as Na or Ca salts, but their detoxification ability in bacteria was unsatisfied (FIG. 2a). Their low activity might be related to either (a) competition in coordination with Pb²⁺ by the two carboxylates, which destabilizes the complexation, or (b) a decrease in their metal selectivity and coordination alkali or alkaline earth metal ions.

Nevertheless, the inventors tested 1a and the other soluble peptides in vitro for their ability to recover poisoned human cells (FIG. 2b). Among all compounds, peptide 8 detoxified Pb². to the greatest extent, with a recovery rate of 334±42%, compared to 110±10% of DMSA and 95±16% of Na₂CaEDTA. This peptide dramatically excelled at high concentrations compared to the benchmark drugs and glutathione (GSH) as a natural reference peptide (FIGS. 2b, c). The inventors also observed similar patterns in most compounds between the two assays, suggesting that the effect of Pb²⁺ on the cellular level and the chelating mechanism are similar in both systems.

The administration of EDTA as a CA has been evolved from its Na salt to Na₂CaEDTA, to decrease undesired depletion of Ca²⁺ ions. Therefore, it was tested whether also in the case of 8, the counter cations affect its activity (FIG. 2d). Unlike EDTA, which shows a higher activity as a Ca-salt, 8 is barely affected by the counter cation (FIG. 2d). These differences hint that the affinity of 8 to Pb²⁺ is higher than Ca²⁺ ions that, unlike with EDTA, do not bind to the CA. The lower activity of Ca8 in high concentrations is associated with a slightly lower solubility of this salt with comparison to Na₂8.

To conclude the effectiveness of 8, the inventors assessed its in vitro toxicity (FIG. 2e) that is dramatically lower than that of DMSA and Na₂CaEDTA, inhibiting the viability of only 15±5% of the population.

Material and Methods

The peptides described herein are synthesized according to the reaction shown in Scheme 2. R depicts for the side chain of an α- or β-amino acid. The side chain may be protected by a suitable protecting group (R′). The tetrapeptide is obtained by standard solid phase peptide synthesis (SPPS) using a standard Fmoc-based protocol on a chlorotrityl chloride resin. Cleavage (1% TFA) is achieved by using TFA in CH₂Cl₂ in 5 rounds of 1 min each. Cyclization is obtained by reacting the sidechain-protected peptide with PyBOP (as a coupling reagent) and Hunig's base (DIPEA; as the base) in a ratio of 1.5 equivalents for PyBOP and 3 equivalents for the base, with respect to the peptide. The peptide is highly diluted (0.1 mM) to avoid dimerization and the solvent is solely CH₂Cl₂. The reaction mixture is incubated overnight (16-48 h). The sidechains are deprotected with a TFA cocktail that is adjusted to the respective amino acid composition.

Typically, a mixture of TFA:TIPS:EDT:H₂O (87.5:2.5:7.5:2.5) is applied for 1 h. Finally, the cyclic tetrapeptide is purified by precipitation in an aqueous solution without the need for HPLC. Purities of 95% and higher and yields in the range of 62% to 87% (after purification) are reached. In a last step, the peptide is reacted with HCl so that Cl⁻ ions replace the TFA anions as TFA is toxic.

The complete removal of TFA is monitored with ¹⁹F NMR.

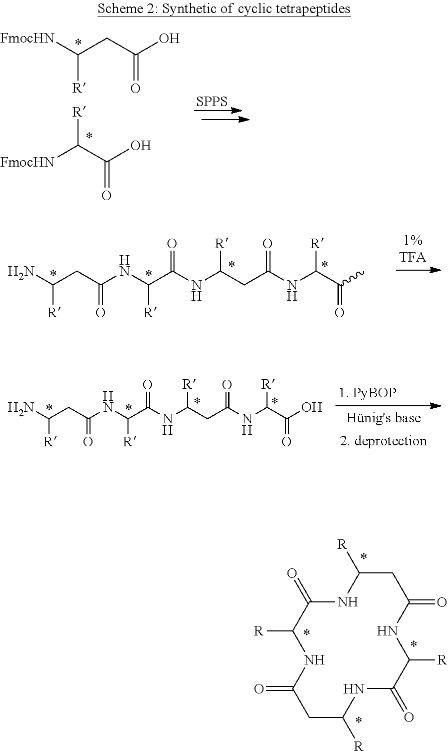

The following cyclic tetrapeptide were synthesized as described above:

| (SEQ ID NO: 1) | |
|---|---|
| | Cys-βAla-Cys-βAla |

HRMS (ESI) m/z calculated for C₁₋₂H₂₁N₄O₄S₂⁺ [M+H]⁺ 349.09987; found: 349.09946

| (SEQ ID NO: 6) | |
|---|---|
| | Cys-βAla-Met-βAla |

HRMS (ESI) m/z calculated for C₁₄H₂₅N₄O₄S₂⁺ [M+H]+ 377.13117; found: 377.13120

| (SEQ ID NO: 4) | |
|---|---|
| | His-βAla-His-βAla |

HRMS (ESI) m/z calculated for C₁₈H₂₆N₆O₄²⁺ [M+2H]². 209.10330; found: 209.10341

| (SEQ ID NO: 7) | |
|---|---|
| | Cys-βAla-His-βAla |

HRMS (ESI) m/z calculated for C₁₅H₂₃N₆O₄²⁺ [M+H]+ 383.14960; found: 383.14971

| (SEQ ID NO: 5) | |
|---|---|
| | Asp-βAla-Asp-βAla |

HRMS (ESI) m/z calculated for C₁₄H₁₉N₄O₈⁻ [M−H]⁻ 371.12084; found: 371.12065

| (SEQ ID NO: 8) | |
|---|---|
| | Cys-βAla-Asp-βAla |

HRMS (ESI) m/z calculated for C₁₋₃H₂₁N₄O₆S⁺ [M+H]⁺ 361.11763; found: 361.11771

| (SEQ ID NO: 2) | |
|---|---|
| | Cys-βAla-DCys-βAla |

HRMS (ESI) m/z calculated for C₁₋₂H₂₁N₄O₄S₂⁺ [M+H]⁺ 349.09987; found: 349.09978

| (SEQ ID NO: 10) | |
|---|---|
| | Cys-βAsp-Cys-βAsp |

HRMS (ESI) m/z calculated for C₁₋₄H₁₉N₄O₈S₂⁻ [M−H]⁻ 435.06498; found: 435.06564

| (SEQ ID NO: 9) | |
|---|---|
| | Cys-βAla-Phe-βAla |

HRMS (ESI) m/z calculated for C₁₈H₂₅N₄O₄S⁺ [M+H]+ 393.15910; found: 393.15888

| (SEQ ID NO: 3) | |
|---|---|
| | Met-βAla-Met-βAla |

HRMS (ESI) m/z calculated for C₁₆H₂₆N₄O₄SNa⁺ [M+Na]⁺ 427.14442; found: 427.14447 In vivo recovery tests A single colony of DH5a E. coli WT cells was grown overnight at 37° C. and 220 rpm in Tris Minimal Medium (TMM, pH 6.0; 5 mL) without antibiotics. The culture was then diluted to an OD₆₀₀ of 0.03 with additional TMM to a total volume of 5 mL, and its OD₆₀₀ was monitored. Upon cell density of 0.25 (which was achieved after 3-5 h), 1 mL of the culture was transferred into a cell culture tube and was labeled as the positive control. To additional 3 mL of the culture, 36 μL of Pb(NO₃)₂ 1 M were added (final concentration of 12 mM). Both cultures were shaken at 37° C. and 220 rpm for an additional 5 h.

Aqueous stock solutions of each CA were plated on freshly prepared agar-LB plates such that the final concentration of each compound is equal to 0.5, 1, 2, 5, and 10 equivalents, compared to the amount of Pb(NO₃)₂ in 50 μL pre-toxified culture. The stock solutions were prepared such that plating and equally spreading 30 μL of each solution will provide the desired amount of CA. To additional two plates, 30 μL of H₂O was added.

50 μL of the pre-toxified culture were homogenously spread to each CA-containing plate, 5 h after adding the metal to the culture. The Pb-containing culture was also plated on one of the two H₂O-containing plates and was labeled as the negative control. Lastly, 50 μL of the non-toxified culture were platted on the second H₂O-containing plate and was labeled as positive control. All plates (positive and negative control and five plates for each examined compound) were then incubated at 37° C. overnight. The plates were then imaged, and the colonies were counted (with Vilber Quantum Visualization System). The recovery of each concentration of CA was calculated according to equation 1:

$$Recovery \% = \frac{\# {CA}_{X}}{\# NEG} \times 100 \%$$

#CAₓ—number of colonies of pre-toxified culture in a plate containing X mM of CA

#NEG—number of colonies of pre-toxified culture in a plate containing no CA

Each experiment was performed on three independent occasions. Values are mean±SD of >3 repeats each performed in triplicate.

In vitro recovery tests

HT-29 cells (purchased from ATCC) were grown in 25 mM HEPES RPMI-1640 medium, supplemented with 1% L-glutamine, 1% penicillin/streptomycin and 10% fetal calf serum (FCS) superior (standardized) at 37° C. and 5% CO₂. 96-well plates were prepared such that every well contains 10,000 cells in 100 μL medium, and the cells were allowed to adhere overnight. To all wells but the positive control, 10 μL of 22 mM Pb(NO₃)₂ were added (final concentration 2 mM). 10 μL of H₂O was added to the positive control wells. 60 min after addition of metal, 10 μL of each solution of the examined CA (2.4, 6, 12, 24, 48, and 120 mM) were added to reach final concentrations of 0.2, 0.5, 1, 2, 4, and 10 mM (which are 0.1, 0.25, 0.5, 1, 2, and 5 equivalents, respectively). To the positive control wells containing no metal and the negative control wells containing metal but no CA, 10 μL of H₂O were added. Each condition was performed in triplicates. The plates were incubated at 37° C. and 5% CO₂ for an additional 23 h, after which the medium was removed, washed with fresh medium and 50 μL of crystal violet solution (0.5% crystal violet powder in 20 mL MeOH and 80 mL H₂O) to each well and the plates were gently shaken (60 rpm) for 20 min. The plates were then washed with H₂O until no more unbound dye was observed and allowed to dry overnight. 200 μL of MeOH were added to each well, and the plates were gently shaken (60 rpm) for 20 min, after which their absorbance at 560 nm was read with a plate reader. The recovery of each concentration of CA was calculated according to equation 2:

$$Recovery \% = \frac{A [ C {A}_{X} ] - A [ blank ]}{A [ NEG ] - A [ blank ]} \times 100 \%$$

A[CAₓ]—absorbance of the pre-toxified well in the presence of X mM of CA

A[blank]—absorbance of blank wells (contain nothing)

A[NEG]—absorbance of the pre-toxified well that contains no CA

Each experiment was performed on three independent occasions. Values are mean±SD of >3 repeats each performed in triplicate.

In Vitro Toxicity Tests

HT-29 cells (purchased from ATCC) were grown in 25 mM HEPES RPMI-1640 medium, supplemented with 1% L-glutamine, 1% penicillin/streptomycin and 10% fetal calf serum (FCS) superior (standardized) at 37° C. and 5% CO₂. 96-well plates were prepared such that every well contains 10,000 cells in 100 μL medium, and the cells were allowed to adhere overnight.

To all wells but the positive control, 10 μL of each solution of the examined CA (2.4, 6, 12, 24, 48, and 120 mM) were added to reach final concentrations of 0.2, 0.5, 1, 2, 4, and 10 mM. To the positive control wells, 10 μL of H₂O was added. Each condition was performed in triplicates. The plates were incubated at 37° C. and 5% CO₂ for 24 h, after which the medium was removed, washed with fresh medium and 50 μL of crystal violet solution (0.5% crystal violet powder in 20 mL MeOH and 80 mL H₂O) to each well and the plates were gently shaken (60 rpm) for 20 min. The plates were then washed with H₂O until no more unbound dye was observed and allowed to dry overnight. 200 μL of MeOH were added to each well, and the plates were gently shaken (60 rpm) for 20 min, after which their absorbance at 560 nm was read with a plate reader. The toxicity of each concentration of CA was calculated according to equation 3:

$$Toxicity \% = \frac{A [ C {A}_{X} ] - A [ blank ]}{A [ POS ] - A [ blank ]} \times 100 \%$$

A[CAₓ]—absorbance of the pre-toxified well in the presence of X mM of CA

A[blank]—absorbance of blank wells (contain nothing)

A[POS]—absorbance of well that contains no CA

Each experiment was performed on three independent occasions. Values are mean±SD of >3 repeats each performed in triplicate.

The set-ups of in vitro and in vivo assays for determination of Pb detoxification ability could be seen in Table 2.

TABLE 2
Set-ups of in vitro and in vivo assays for determination of Pb detoxification ability

| | In vitro assay | In vivo assay |
|---|---|---|
| Cells | HT-29^{a} | DH5α E. coli^{b} |
| Cell density at t = 0 | 10,000 cells per well | OD₆₀₀ 0.25 |
| [Pb(NO₃)₂] (mM) | 2^{c} | 12^{c} |
| Incubated time with Pb²⁺ (h) | 1 | 5 |
| Concentrations of compounds (mM) | 0-10^{d} | 0-120^{e} |
| Incubation with compounds | Overnight, 37° C., 5% CO₂ | Overnight, 37° C., 220 rpm |
| Further steps | Washing with fresh medium | Plating on LB agar |
| Positive control | Non-poisoned cells | Non-poisoned cells |
| Negative control | Untreated cells | Untreated cells |
| Detection of viability | Crystal violet^{f} | Colony counting^{g} |

^{a}HT-29 cells were chosen as model human cells since they show high sensitivity to Pb, and the latter does not precipitate in their medium (RPMI-1640);
^{b}DH5α E. coli strain was chosen due to its low minimal inhibitory concentration of Pb;
^{c}The lowest concentration that shows a significant effect at the shortest timeframe;
^{d}0-5 equiv.;
^{e}0-10 equiv.;
^{f}Dyes based on reduction (such as MTT) cannot be of use due to competing reduction by thiols;
^{g}Due to Pb precipitation over time, colonies counting was found to be more accurate and indicative than optical density.

### Example 3: Peptide 8a

In Vitro and In Vivo Detoxification Results

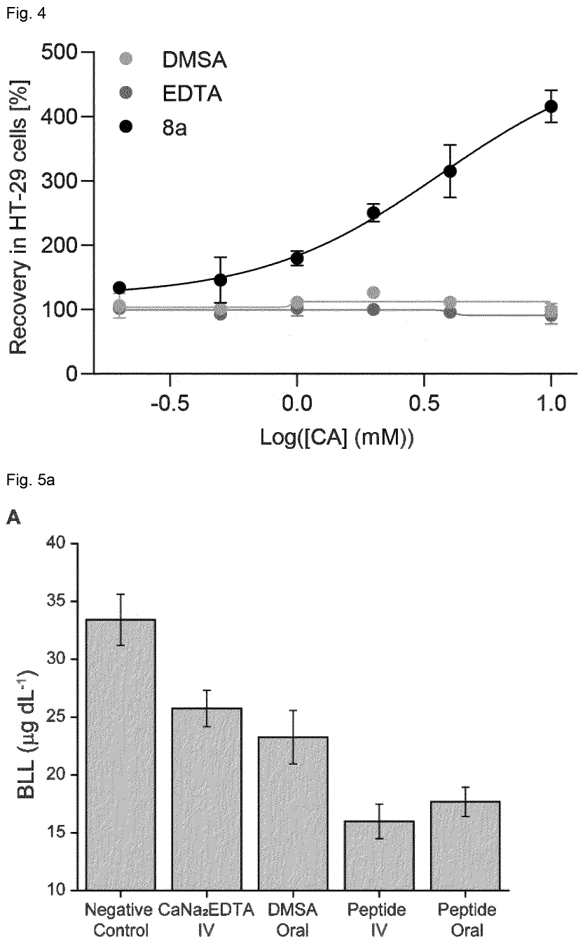

The peptide that revealed the best results within all investigated peptides and also outcompeted the standards of care (SOCs) (FIG. 4) has the sequence cyc-[SAsp-βAla-Asp-βAla] (8a; SEQ ID NO: 16):

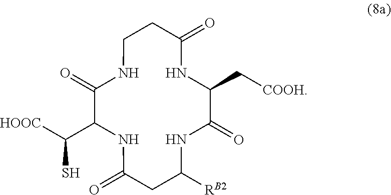

Peptide 8a was then tested in mice. 40 male mice (C₅₇BL/6) aged 6-8 weeks were provided with 20 mM Pb(OAc)₂ solution as the sole water supply for seven days (days 1-7). This poisoning route mimics chronic exposure in humans. Two days after returning to clean water (day 9), they were randomly divided into five groups of eight mice. They received a single treatment per day for seven days (days 10-16) of either CaNa₂EDTA, DMSA, or 8a at a concentration of 30 mg kg⁻¹ except for group 1 that served as the negative control (Table 3).

Blood samples (100 μL) were collected before dosing the mice on days 10-15 and on day 18, which is two days after the last dosing and the day by which the experiment was terminated. Urine was also collected from 34 animals on day 18 and was kept frozen until analysis.

TABLE 3
The treatments administered to each group of eight mice and the quantified values

| | | | Dose | BLL | Urinary Pb |
|---|---|---|---|---|---|
| Group | Treatment | Administration | (mg kg⁻¹ day⁻¹) | (μg dL⁻¹) | (ppm) |
| 1 | None | — | — | 33.4 ± 2.2 | 154 ± 38 |
| 2 | CaNa₂EDTA | IV | 30 | 25.7 ± 1.6 | 210 ± 30 |
| 3 | DMSA | Oral | | 23.3 ± 2.3 | 340 ± 28 |
| 4 | 8a | IV | | 16.0 ± 1.5 | 452 ± 47 |
| 5 | | Oral | | 17.7 ± 1.3 | 435 ± 28 |

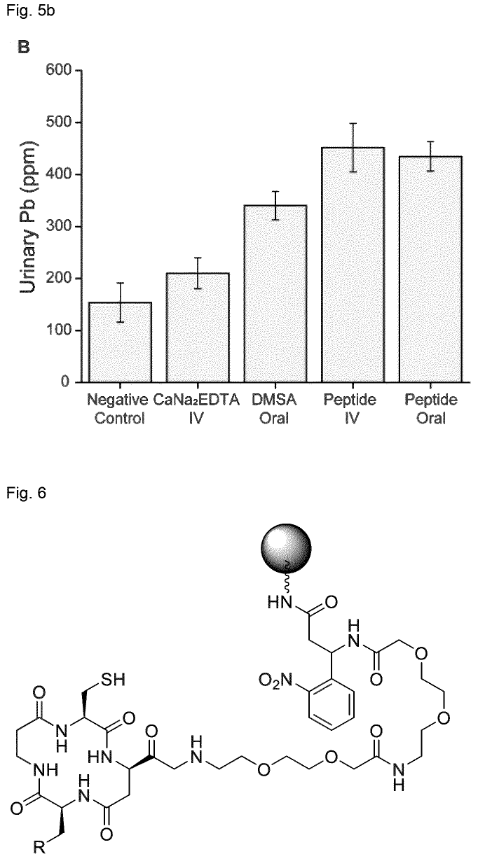

The blood samples from the last day clearly indicate that 8a is more efficient than the two SOCs, both when administered orally and IV (Table 3, FIG. 5a). Specifically, while given IV, 8a reduced the average blood lead levels (BLL) by 2.1 folds compared to no treatment and 1.6 folds compared with CaNa₂EDTA, which is also given IV. When given orally, the peptide reduced the BLL by 1.9 folds compared with no treatment and 1.3 folds compared with DMSA.

The Pb content in the urine of 34 mice (out of 40; FIG. 5b) collected during the last day of the experiment indicates that the mechanism of action of 8a is by chelation and expulsion of the toxic metal via the urine. The high Pb levels in the urine of groups 4 and 5 align with the reduced BLL of these groups compared with groups 1-3. Comparing the IV and oral administration of the peptide, Pb was expelled at 2.9 and 2.8 folds compared with the untreated group, respectively. The peptide also enabled elevated removal of Pb compared with the SOCs in the range of 1.3-2.2 folds.

Water remediation with immobilized peptides Two peptides that are expected to tightly and selectively bind Pb²⁺ ions were immobilized to a solid support with a long and flexible linker ((PEG2)₂) and a photocleavable moiety (FIG. 6).

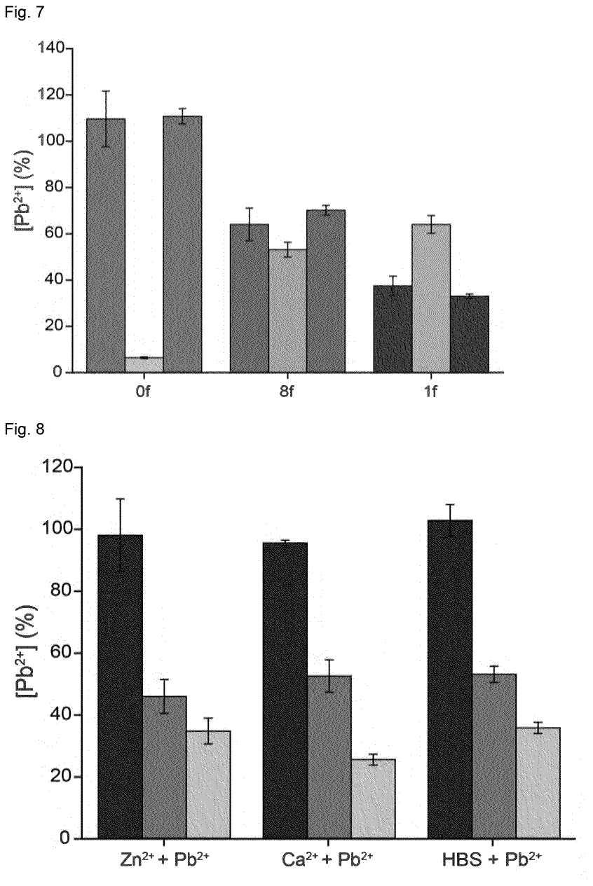

In addition, a negative control by which the second PEG2 is acetylated (Of) was synthesized. All three devices were then tested for their ability to capture Pb²⁺ ions from a 25 mM Pb(NO₃)₂ solution. 1 h after adding the metal solution to the devices, the solutions were filtered, and the Pb concentration in each of them was quantified by ICP-MS. The efficacy was calculated by dividing the concentration of each solution by the concentration detected in the original solution as the 100% Pb content (FIG. 7).

While 0f was incapable of reducing Pb-concentration of the contaminated solution, 1f and 8f reduced Pb concentration by 62±4% and 36±7%, respectively (FIG. 7, left dark gray bars), indicating their efficacy in removing Pb²⁺ ions from aqueous solutions.

The resins were then treated with 100 mM Na₂EDTA solutions for 10 min and quantified their Pb concentration to indicate an effective resin regeneration (FIG. 7 light gray bars). The filtration experiment was then repeated (FIG. 7, dark gray right bars) and showed similar results to the first round, revealing the possibility to recover the resin by washing Pb out with a cost-efficient EDTA solution.

To detect the metal selectivity of our devices, similar filtration experiments were performed in equimolar mixtures of ZnCl₂+Pb(NO₃)₂ and CaCl₂+Pb(NO₃)₂ and in human blood serum (HBS) that was spiked with 25 mM of Pb(NO₃)₂(FIG. 8). The Pb concentrations, as well as the Ca or Zn concentrations of the first two experiments, revealed that 1f and 8f do not capture these essential metals as marginal amounts of Zn and Ca were detected in the filtrates. Noteworthy, these devices removed Pb to a similar extent as in the first experiment where no additional metal salts were present, indicating the metal selectivity of our devices. Similar results were also achieved in the Pb-spiked HBS (FIG. 8).

### LIST OF REFERENCES

Sears, Margaret E. Chelation: Harnessing and Enhancing Heavy Metal Detoxification—A Review. 2013. The Scientific World Journal, Volume 2013, Article ID 219840, 13 pages
White, C. J.; Yudin, A. K. Contemporary Strategies for Peptide Macrocyclization. Nat. Chem. 2011, 3 (7), 509-524. https-J/doi.org/10.1038/nchem.1062.
Feoktistova, M.; Geserick, P.; Leverkus, M. Crystal Violet Assay for Determining Viability of Cultured Cells. Cold Spring Harb. Protoc. 2016, 2016 (4), 343-346. httpsJ/doi.org/10.1101/pdb.prot087379.

1. A compound having the structure of formula 1; 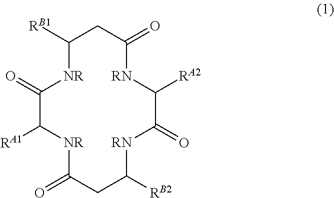
(1a), wherein
each R independently from any other R is independently selected from —CH₃ and —H,
R^{A1} and R^{A2} are independently from each other a C₁₋₄-alkyl or phenyl, wherein the C₁₋₄-alkyl or phenyl is substituted by one or more substituents independently selected from —SH, —S—C₁₋₄-alkyl, —SeH, —Se—C₁₋₄-alkyl, —SO₃H, —COOH, —NH₂, —CONH₂, —NH—C(═NH)(NH₂) a 5- to 10-membered heterocycle, a cyclic hydrocarbon moiety comprising 3 to 10 carbon atoms, wherein
the 5- to 10-membered heterocycle or the cyclic hydrocarbon moiety may optionally be substituted by one or more substituents selected from C₁₋₄-alkyl, —SH, (═S), —S—C₁₋₄-alkyl, —SeH, —Se—C₁₋₄-alkyl, —SO₃H, (═O), —COOH, —NH₂, —CONH₂,
R^{B1} and R^{B2} are independently from each other;
—H, or
a moiety selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a 5- to 10-membered heterocycle or a hydrocarbon moiety comprising 1 to 12 C atoms, wherein
the 5- to 10-membered heterocycle or the hydrocarbon moiety is optionally substituted by one or more substituents independently selected from —OH, (═O), —SH, (═S), —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle, or
a linker suitable for binding to a detectable marker or a solid support,
a detectable marker, optionally linked by a linker, or
a linker bound to a solid support.

2. The compound according to claim 1, wherein the compound has the structure of formula 2, 3, 4, 5, 6 or 7

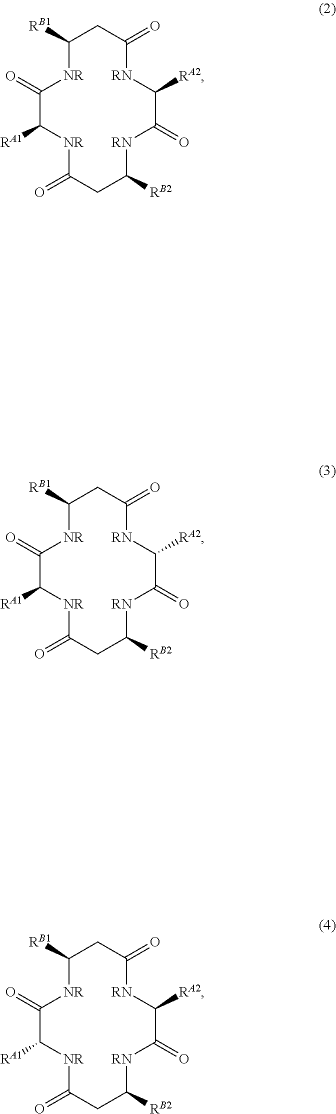

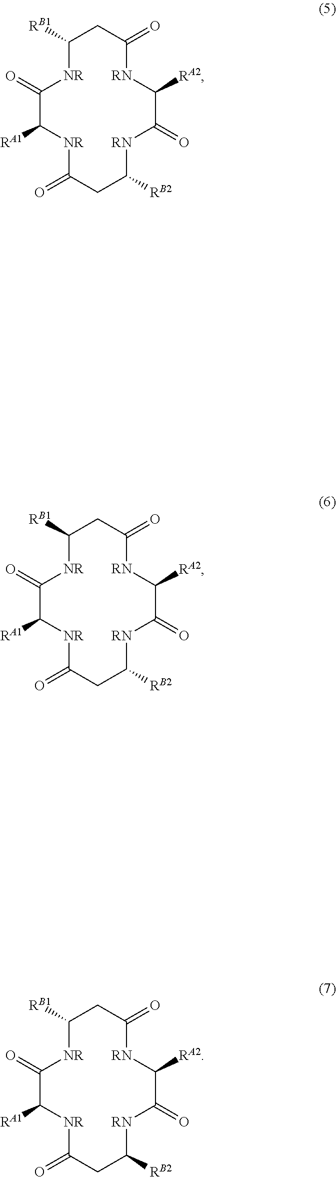

3. The compound according to claim 1, wherein R^{A1} and R^{A2} are independently from each other a C₁₋₄-alkyl, substituted by one or more substituents independently selected from —SH, —S—C₁₋₄-alkyl, —SeH, —Se—C₁₋₄-alkyl, —SO₃H, —COOH, —NH₂, —CONH₂, a five- to 10-membered heterocycle, a cyclic hydrocarbon moiety comprising 3 to 6 carbon atoms, wherein
the 5- to 10-membered heterocycle or the cyclic hydrocarbon moiety may optionally be substituted by one or more substituents selected from C₁₋₄-alkyl, —SH, (═S), —S—C₁₋₄-alkyl, —SeH, —Se—C₁₋₄-alkyl, —SO₃H, (═O), —COOH, —NH₂, —CONH₂.

4. The compound according to claim 1, wherein the cyclic hydrocarbon moiety at R^{A1} and R^{A2} is selected from cyclopentyl, cyclohexyl, and phenyl.

5. The compound according to claim 1, wherein R^{A1} and R^{A2} are independently from each other a C₁₋₃-alkyl, substituted by 1 or 2 substituents independently selected from —SH, —S—CH₃, —SeH, —Se—CH₃, —SO₃H, —COOH, —NH₂, —CONH₂, imidazolyl, mercaptoimidazolyl, thiofuranyl, indolyl, and phenyl, wherein
the phenyl may optionally be substituted by one or more substituents selected from —SH, and —SeH.

6. The compound according to claim 1, wherein R^{A1} and R^{A2} are independently selected from —CH₂—SH, —(CH₂)₂—SH, —CH₂—S—CH₃, —(CH₂)₂—S—CH₃, —CH(SH)(—CH₂—SH), —CH₂—CH(SH)(—CH₂—SH), —CH(SH)(—COOH), —CH(SH)—CH₂—COOH, —CH₂—CH(SHX—COOH), -phenyl-SH, —CH₂—SO₃H, —(CH₂)₂—SO₃H —CH₂—COOH, —(CH₂)₂—COOH, —CH₂—NH₂, —(CH₂)₂—NH₂, —CH₂—CONH₂, —(CH₂)₂—CONH₂, —CH₂— imidazolyl, —CH₂-mercaptoimidazolyl, and —CH₂-phenyl.

7. The compound according to claim 1, wherein R^{B1} and R^{B2} are independently selected from
—H, or
a moiety selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a 5- to 10-membered heterocycle or a hydrocarbon moiety comprising 1 to 12 C atoms, wherein
the 5- to 10-membered heterocycle or the hydrocarbon moiety is optionally substituted by one or more substituents independently selected from —OH, (═O), —SH, (═S), —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle.

8. The compound according to claim 1, wherein R^{B1} and R^{B2} are independently selected from
—H, or
a moiety selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, a 5- to 10-membered heterocycle, a cyclopentyl, a cyclohexyl, phenyl or a C₁₋₄-alkyl, wherein
the cyclopentyl, a cyclohexyl, phenyl or the C₁₋₄-alkyl is optionally substituted by one or more substituents independently selected from —OH, —SH, —S—C₁₋₄-alkyl, —SeH, —Se—C₁₋₄-alkyl, —COOH, —NH₂, —NH—C₁₋₄-alkyl, —NH—C(═NH)(NH₂), —CONH₂, —SO₃H, and a five- to 10-membered heterocycle.

9. The compound according to claim 1, wherein R^{B1} and R^{B2} are independently selected from H, —C₃₋₆-alkyl, —CH₂-phenyl, —SH, —(CH₂)ₘ—SH, —(CH₂)ₘ—COOH and —(CH₂)ᵣ—CONH₂ with m and r being 0, 1, 2 or 3.

10. The compound according to claim 1, wherein the heterocycle at R^{A1} and R^{A2} and/or at R^{B}I and R^{B2} is selected from piperidinyl, piperazinyl, pyridinyl, pyrimidinyl, pyrazinyl, pyridazinyl, pyrrolyl, pyrazolyl, imidazolyl, mercaptoimidazolyl, thiofuranyl, oxazolonyl, indolyl, mercaptopurinyl, and benzothiophenyl.

11. The compound according to claim 1, wherein R^{A1} and R^{A2} are identical and/or R^{B1} and R^{B2} are identical.

12. The compound according to claim 1, wherein
the detectable marker is selected from a dye, an affinity tag, a magnetic bead and a moiety comprising a radioisotope, and/or
the linker is a hydrocarbon moiety comprising up to 50 C atoms, wherein one or more C atoms may optionally be replaced by O, S or N, and/or
the solid support is a resin, a bead, a surface of an electrode or the bottom/wall of a reaction vessel.

13. A metal complex consisting of a ligand and a metal, wherein the ligand is a compound according to claim 1.

14. A method of treating metal poisoning, comprising administering an amount effective of compound according to claim 1 to a person in need thereof.

15. A method of removing and/or detecting a metal from/in a substrate, wherein the method comprises applying the compound according to claim 1 to the substrate.

16. The compound according to claim 1, wherein the compound has the structure of Formula 1a: [chemical structure]


SEQUENCE LISTING (16 sequences)

SEQ ID NO: 1 - protein, 4 aa, Artificial Sequence
  tetrapeptide
  MISC_FEATURE (2)..(2): beta-Alanin
  MISC_FEATURE (4)..(4): beta-Alanin
CACA

SEQ ID NO: 2 - protein, 4 aa, Artificial Sequence
  tetrapeptide
  MISC_FEATURE (2)..(2): beta-Alanine
  MISC_FEATURE (3)..(3): D-Cysteine
  MISC_FEATURE (4)..(4): beta-Alanine
CACA

SEQ ID NO: 3 - protein, 4 aa, Artificial Sequence
  tetrapeptide
  MISC_FEATURE (2)..(2): beta-Alanine
  MISC_FEATURE (4)..(4): beta-Alanine
MAMA

SEQ ID NO: 4 - protein, 4 aa, Artificial Sequence
  tetrapeptide
  MISC_FEATURE (2)..(2): beta-Alanine
  MISC_FEATURE (4)..(4): beta-Alanine
HAHA

SEQ ID NO: 5 - protein, 4 aa, Artificial Sequence
  tetrapeptide
  MISC_FEATURE (2)..(2): beta-Alanine
  MISC_FEATURE (4)..(4): beta-Alanine
DADA

SEQ ID NO: 6 - protein, 4 aa, Artificial Sequence
  tetrapeptide
  MISC_FEATURE (2)..(2): beta-Alanine
  MISC_FEATURE (4)..(4): beta-Alanine
CAMA

SEQ ID NO: 7 - protein, 4 aa, Artificial Sequence
  tetrapeptide
  MISC_FEATURE (2)..(2): beta-Alanine
  MISC_FEATURE (4)..(4): beta-Alanine
CAHA

SEQ ID NO: 8 - protein, 4 aa, Artificial Sequence
  tetrapeptide
  MISC_FEATURE (2)..(2): beta-Alanine
  MISC_FEATURE (4)..(4): beta-Alanine
CADA

SEQ ID NO: 9 - protein, 4 aa, Artificial Sequence
  tetrapeptide
  MISC_FEATURE (2)..(2): beta-Alanine
  MISC_FEATURE (4)..(4): beta-Alanine
CAFA

SEQ ID NO: 10 - protein, 4 aa, Artificial Sequence
  tetrapeptide
  MISC_FEATURE (2)..(2): beta-Aspartic acid
  MISC_FEATURE (4)..(4): beta-Aspartic acid
CDCD

SEQ ID NO: 11 - protein, 4 aa, Artificial Sequence
  tetrapeptide
  MISC_FEATURE (2)..(2): beta-homoGlutamic acid
  MISC_FEATURE (4)..(4): beta-homoGlutamic acid
CECE

SEQ ID NO: 12 - protein, 4 aa, Artificial Sequence
  tetrapeptide
  MISC_FEATURE (1)..(1): alpha-amino acid
  MISC_FEATURE (1)..(4): head-to-tail cyclization
  MISC_FEATURE (2)..(2): beta-amino acid
  MISC_FEATURE (3)..(3): alpha-amino acid
  MISC_FEATURE (4)..(4): beta-amino acid
XXXX

SEQ ID NO: 13 - protein, 4 aa, Artificial Sequence
  tetrapeptide
  MISC_FEATURE (1)..(1): beta-amino acid
  MISC_FEATURE (2)..(2): alpha-amino acid
  MISC_FEATURE (3)..(3): beta-amino acid
  MISC_FEATURE (4)..(4): alpha-amino acid
XXXX

SEQ ID NO: 14 - protein, 4 aa, Artificial Sequence
  tetrapeptide
  MISC_FEATURE (1)..(1): alpha-amino acid
  MISC_FEATURE (2)..(2): beta-amino acid
  MISC_FEATURE (3)..(3): alpha-amino acid
  MISC_FEATURE (4)..(4): beta-amino acid
XXXX

SEQ ID NO: 15 - protein, 4 aa, Artificial Sequence
  tetrapeptide
  MISC_FEATURE (1)..(1): alpha-amino acid
  MISC_FEATURE (1)..(4): head-to-tail cyclization
  MISC_FEATURE (2)..(2): beta-Alanine
  MISC_FEATURE (3)..(3): alpha-amino acid
  MISC_FEATURE (4)..(4): beta-Alanine
XAXA

SEQ ID NO: 16 - protein, 4 aa, Artificial Sequence
  tetrapeptide
  misc_feature (1)..(1): Aspartic acid substituted by -SH
  misc_feature (1)..(4): head-to-tail cyclization
  misc_feature (2)..(2): beta-Alanine
  misc_feature (4)..(4): beta-Alanine
XADA